(19) United States
(12) Patent Application Publication
(10) Pub. No.: US 2017/0128448 A1
(43) Pub. Date: May 11, 2017

(54) COMBINATION THERAPY FOR CANCER

(71) Applicant: Celgene Corporation, Summit, NJ (US)

(72) Inventors: Michael Pourdehnad, San Francisco, CA (US); Anita Gandhi, Bernardsville, NJ (US); Kenichi Takeshita, New York, NY (US); Rajesh Chopra, Summit, NJ (US)

(21) Appl. No.: 15/323,173

(22) PCT Filed: Jul. 10, 2015
(86) PCT No.: PCT/US15/39939

§ 371 Date: Dec. 30, 2016

Related U.S. Application Data
(60) Provisional application No. 62/023,748, filed on Jul. 11, 2014.
(60) Provisional application No. 62/033,062, filed on Aug. 4, 2014.
(60) Provisional application No. 62/033,566, filed on Aug. 5, 2014.
(60) Provisional application No. 62/149,941, filed on Apr. 20, 2015.
(60) Provisional application No. 62/156,928, filed on May 5, 2015.

(51) Int. Cl. A61K 31/517; C07K 16/28; A61K 39/395

(52) U.S. Cl. CPC A61K 31/517; A61K 39/39558; C07K 16/2887; A61K 2039/545

(57) ABSTRACT

Provided herein are methods of treating, preventing and/or managing lymphomas and leukemias by administering to a patient Compound A (3-(5-amino-2-methyl-4-oxo-4H-quinazolin-3-yl)-piperidine-2,6-dione), or a pharmaceutically acceptable salt, solvate, or stereoisomer thereof, in combination with an anti-CD20 antibody or ibrutinib, or a pharmaceutically acceptable salt or solvate thereof

### CROSS-REFERENCE TO RELATED APPLICATIONS

This application claims the benefit of the filing date of U.S. Provisional Application No. 62/023,748, filed Jul. 11, 2014; U.S. Provisional Application No. 62/033,062, filed Aug. 4, 2014; U.S. Provisional Application No. 62/149,941, filed Apr. 20, 2015; and U.S. Provisional Application No. 62/156,928, filed May 5, 2015, each entitled “Combination Therapy Cancer.” These entire disclosures are hereby incorporated by reference into the present disclosure.

### 1. FIELD

Provided herein are methods of treating, preventing and/or managing lymphomas and leukemias by administering to a patient Compound A (3-(5-amino-2-methyl-4-oxo-4H-quinazolin-3-yl)-piperidine-2,6-dione), or a pharmaceutically acceptable salt, solvate, or stereoisomer thereof, in combination with an anti-CD20 antibody or ibrutinib, or a pharmaceutically acceptable salt or solvate thereof.

### 2. BACKGROUND

The incidence of lymphoma and leukemia, including non-Hodgkin's lymphoma and chronic lymphocytic leukemia continues to climb. A tremendous demand therefore exists for new methods and compositions that can be used to treat patients with lymphomas and leukemias.

Despite availability of a variety of chemotherapeutic agents, chemotherapy has many drawbacks. Stockdale, Medicine, vol. 3, Rubenstein and Federman, eds., ch. 12, sect. 10, 1998. Almost all chemotherapeutic agents are toxic, and chemotherapy causes significant and often dangerous side effects. Moreover, cells can develop resistance to chemotherapeutic agents and can in certain instances develop broadened resistance to other chemotherapeutic agents. Because of the drug resistance, many lymphomas and leukemias relapse or become refractory (relapse/refractory also referred to as “r/r”) to standard chemotherapeutic treatment protocols.

Chemotherapeutic agents can be administered in a combination therapy (e.g., two or more different chemotherapeutic agents) to combat drug resistance and to increase efficacy. There is a need in the art for therapies, both single and combination therapies, for treating r/r lymphomas and r/r leukemias. Provided herein are solutions to these and other problems in the art by providing combination therapies that include Compound A to treat lymphomas and leukemias and r/r lymphomas and leukemias.

### 3. SUMMARY

Provided herein are methods of treating, preventing, or managing lymphoma or leukemia. In one aspect is a method of treating, preventing, or managing lymphoma or leukemia by administering to a subject in need thereof Compound A:

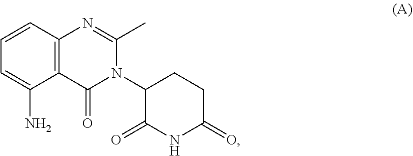 [SMILES: Cc1nc2cccc(N)c2c(=O)n1C1CCC(=O)NC1=O]

or a pharmaceutically acceptable salt, solvate, or stereoisomer thereof, in combination with an anti-CD20 antibody.

Further provided herein is a method of treating, preventing, or managing lymphoma or leukemia by administering to a subject in need thereof Compound A, or a pharmaceutically acceptable salt, solvate, or stereoisomer thereof, in combination with ibrutinib, or a pharmaceutically acceptable salt or solvate thereof.

In another aspect are pharmaceutical compositions, dosage forms, and dosing regimens in connection with the above-described methods. In some embodiments, the pharmaceutical compositions may be prepared as medicaments for the treatment of any of the conditions described herein. In some embodiments, the pharmaceutical compositions may be prepared as medicaments for the prevention of any of the conditions described herein. In some embodiments, the pharmaceutical compositions may be prepared as medicaments for the management of any of the conditions described herein.

### 4. DETAILED DESCRIPTION

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Any methods, devices and materials similar or equivalent to those described herein can be used in the practice of this invention. The following definitions are provided to facilitate understanding of certain terms used frequently herein and are not meant to limit the scope of the present disclosure. All references referred to herein are incorporated by reference in their entirety.

The terms “3-(5-amino-2-methyl-4-oxo-4H-quinazolin-3-yl)-piperidine-2,6-dione” and “Compound A” are used interchangeably herein and refer to a compound having structure:

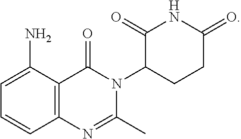 [SMILES: Cc1nc2cccc(N)c2c(=O)n1C1CCC(=O)NC1=O]

Compound A also refers to enantiomers or a mixture of enantiomers thereof, pharmaceutically acceptable stereoisomers, pharmaceutically acceptable salts, prodrugs, solvates, hydrates, co-crystals, clathrates, or polymorphs thereof. In certain embodiments, Compound A refers to the base and pharmaceutically acceptable salts thereof.

As used herein, unless otherwise specified, the terms “treat,” “treating,” and “treatment” refer to alleviating or abrogating a disease, e.g., lymphoma or leukemia, or one or more of the symptoms associated with the disease; or alleviating or eradicating the cause(s) of the disease itself.

As used herein, unless otherwise specified, the term “preventing” refers to the treatment with or administration of a compound provided herein, with or without another additional active compound, prior to the onset of symptoms, particularly to patients at risk of lymphoma or leukemia and/or other disorders described herein. The term “prevention” includes the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

As used herein, and unless otherwise specified, the terms “manage,” “managing” and “management” refer to preventing or slowing the progression, spread or worsening of a disease or disorder, (e.g., lymphoma or leukemia) or of one or more symptoms thereof. In certain cases, the beneficial effects that a subject derives from a prophylactic agent do not result in a cure of the disease or disorder. In certain cases, the beneficial effects that a subject derives from a therapeutic agent do not result in a cure of the disease or disorder.

The terms “subject” and “patient” refer to an animal, including, but not limited to, a mammal, including a primate (e.g., human), cow, sheep, goat, horse, dog, cat, rabbit, rat, or mouse. The terms “subject” and “patient” are used interchangeably herein in reference, for example, to a mammalian subject, such as a human subject. In various embodiments, subjects herein can be characterized by the disease being treated (e.g., a “lymphoma subject” or a “leukemia subject”).

As used herein, unless otherwise specified, the term “therapeutically effective amount” of a compound refers to the amount of a compound that, when administered, is sufficient to prevent development of, or alleviate to some extent, one or more of the symptoms of a disease, e.g., lymphoma or leukemia, being treated. The term also refers to the amount of a compound that is sufficient to elicit the biological or medical response of a biological molecule (e.g., a protein, enzyme, RNA, or DNA), cell, tissue, system, animal, or human that is being sought by a researcher, veterinarian, medical doctor, or clinician. Furthermore, a therapeutically effective amount of a compound means an amount of a therapeutic agent, alone or in combination with other therapies that provides a therapeutic benefit in the treatment or management of a disease, e.g., lymphoma or leukemia. The term encompasses an amount that improves overall therapy, reduces, or avoids symptoms or causes of a disease, e.g., lymphoma or leukemia, or enhances the therapeutic efficacy of another therapeutic agent.

As used herein, and unless otherwise specified, a “prophylactically effective amount” of a compound is an amount sufficient to inhibit or reduce a symptom of lymphoma or leukemia or to prevent recurrence of lymphoma or leukemia. A prophylactically effective amount of a compound means an amount of therapeutic agent, alone or in combination with other agents that provides a prophylactic benefit in the inhibition or reduction of a symptom of lymphoma or leukemia or recurrence of lymphoma or leukemia. The term “prophylactically effective amount” can encompass an amount that improves overall prophylaxis or enhances the prophylactic efficacy of another prophylactic agent.

As used herein, “administer” and “administration” refer to the act of physically delivering a substance as it exists outside the body into a subject. Administration includes all forms known in the art for delivering therapeutic agents, including but not limited to oral, topical, mucosal, injections, intradermal, intravenous, intramuscular delivery or other method of physical delivery described herein or known in the art (e.g., implantation of a slow-release device, such as a mini-osmotic pump to a subject; liposomal formulations; buccal; sublingual; palatal; gingival; nasal; vaginal; rectal; intra-arteriole; intraperitoneal; intraventricular; intracranial; or transdermal). Preferably, the compositions (e.g., Compound A) described herein are administered orally (e.g., by capsule or tablet).

By “co-administer” it is meant that a composition described herein is administered at the same time, just prior to, or just after the administration of one or more additional therapeutic compositions, including, for example, an anti-CD20 antibody or ibrutinib. The terms “co-administration,” “in combination with,” and grammatical equivalents thereof are used interchangeably herein. Co-administration is meant to include simultaneous or sequential administration of a compound individually or in combination (more than one compound or agent). Co-administration includes administering two active agents simultaneously, approximately simultaneously (e.g., within about 1, 5, 10, 15, 20, or 30 minutes of each other), or sequentially in any order. Thus, co-administration can include administering one active agent (e.g. a compound described herein) within 0.5, 1, 2, 4, 6, 8, 10, 12, 16, 20, or 24 hours of a second active agent. Co-administration can also be accomplished by co-formulation, e.g., preparing a single dosage form including both active agents. The active agents can be formulated separately. In such instances, the active agents are admixed and included together in the final form of the dosage unit. Alternatively, co-administration as described herein can include administering two separate unit dosage forms of at least two separate active agents (e.g., Compound A, or an enantiomer or a mixture of enantiomers thereof; or a pharmaceutically acceptable salt, solvate, hydrate, co-crystal, clathrate, or polymorph thereof and a second active agent described herein). The term encompasses administration of two or more agents to a subject so that both agents and/or their metabolites are present in the subject at the same time. Co-administration includes simultaneous administration in separate compositions, administration at different times in separate compositions, or administration in a composition in which both agents are present.

As used herein, the term “daily” is intended to mean that a therapeutic compound, such as Compound A, is administered once or more than once each day for a period of time. The term “continuous” is intended to mean that a therapeutic compound, such as Compound A, is administered daily for an uninterrupted period of at least 10 days to 52 weeks. The terms “intermittent” and “intermittently” as used herein are intended to mean stopping and starting at either regular or irregular intervals. For example, intermittent administration of Compound A, or a pharmaceutically acceptable stereoisomer, a pharmaceutically acceptable salt, prodrug, solvate, hydrate, co-crystal, clathrate, or polymorph thereof is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as Compound A, is administered daily or continuously but with a rest period.

“Lymphoma” refers to cancers that originate in the lymphatic system. Lymphoma is characterized by malignant neoplasms of lymphocytes—B lymphocytes and T lymphocytes (i.e., B-cells and T-cells). Lymphoma generally starts in lymph nodes or collections of lymphatic tissue in organs including, but not limited to, the stomach or intestines. Lymphoma may involve the marrow and the blood in some cases. Lymphoma may spread from one site to other parts of the body.

The treatment of various forms of lymphomas are described, for example, in U.S. Pat. No. 7,468,363, the entirety of which is incorporated herein by reference. Examples of lymphomas include, but are not limited to, Hodgkin's lymphoma, non-Hodgkin's lymphoma, cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma and low grade follicular lymphoma.

“Non-Hodgkin's lymphoma” and “NHL” are used interchangeably herein and refer to malignant monoclonal proliferation of lymphoid cells in sites of the immune system, including lymph nodes, bone marrow, spleen, liver, and gastrointestinal tract. Examples of types of NHL include, but are not limited to, mantle cell lymphoma (MCL), lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), follicular lymphoma (FL), and types of mantle cell lymphomas that can be seen under the microscope (e.g., nodular, diffuse, blastic or mantle zone lymphoma).

NHL represents the fifth most common cancer for both men and women in the United States, with an estimated 63,190 new cases and 18,660 deaths in 2007. Jemal A, et al., CA Cancer J Clin 2007; 57(1):43-66. The probability of developing NHL increases with age and the incidence of NHL in the elderly has been steadily increasing in the past decade, causing concern with the aging trend of the US population. Id. Clarke C A, et al., Cancer 2002; 94(7):2015-2023.

“Diffuse large B-cell lymphoma” or “DLBCL” refers to a fast-growing (i.e., aggressive) type of NHL and accounts for approximately one-third of non-Hodgkin's lymphomas. Diffuse large B-cell lymphoma (DLBCL) can affect any age group but occurs mostly in older people (the average age is mid-60s). It usually starts as a quickly growing mass in a lymph node deep inside the body, such as in the chest or abdomen, or in a lymph node you can feel, such as in the neck or armpit. It can also start in other areas such as the intestines, bone, or even the brain or spinal cord. Anticancer drugs cause rapid and persistent depletion of lymphocytes, possibly by direct apoptosis induction in mature T and B cells. See K. Stahnke. et al., Blood 2001, 98:3066-3073. Absolute lymphocyte count (ALC) has been shown to be a prognostic factor in follicular non-Hodgkin's lymphoma and recent results have suggested that ALC at diagnosis is an important prognostic factor in diffuse large B-cell lymphoma. See D. Kim et al., Journal of Clinical Oncology, 2007 ASCO Annual Meeting Proceedings Part I. Vol 25, No. 18S (June 20 Supplement), 2007: 8082.

“Follicular non-Hodgkin's lymphoma” refers to a slow growing (i.e., indolent) form of NHL.

“Leukemia” refers to malignant neoplasms of the blood-forming tissues. Leukemia is generally clinically classified on the basis of: (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Various forms of leukemias are described, for example, in U.S. Pat. No. 7,393,862 and U.S. provisional patent application No. 60/380,842, filed May 17, 2002, the entireties of which are incorporated herein by reference. Although viruses reportedly cause several forms of leukemia in animals, causes of leukemia in humans are to a large extent unknown. The Merck Manual, 944-952 (17^{th} ed. 1999). Transformation to malignancy typically occurs in a single cell through two or more steps with subsequent proliferation and clonal expansion. In some leukemias, specific chromosomal translocations have been identified with consistent leukemic cell morphology and special clinical features (e.g., translocations of 9 and 22 in chronic myelocytic leukemia, and of 15 and 17 in acute promyelocytic leukemia). Acute leukemias are predominantly undifferentiated cell populations and chronic leukemias are more mature cell forms.

Acute leukemias are divided into lymphoblastic (ALL) and non-lymphoblastic (ANLL) types. The Merck Manual, 946-949 (17^{th} ed. 1999). They may be further subdivided by their morphologic and cytochemical appearance according to the French-American-British (FAB) classification or according to their type and degree of differentiation. The use of specific B- and T-cell and myeloid-antigen monoclonal antibodies are most helpful for classification. ALL is predominantly a childhood disease that is established by laboratory findings and bone marrow examination. ANLL, also known as acute myelogenous leukemia or acute myeloblastic leukemia (AML), occurs at all ages and is the more common acute leukemia among adults; it is the form usually associated with irradiation as a causative agent.

Chronic leukemias are described as being lymphocytic (CLL) or myelocytic (CML). The Merck Manual, 949-952 (17^{th} ed. 1999). CLL is characterized by the appearance of mature lymphocytes in blood, bone marrow, and lymphoid organs. The hallmark of CLL is sustained, absolute lymphocytosis (>5,000/μL) and an increase of lymphocytes in the bone marrow. Most CLL patients also have clonal expansion of lymphocytes with B-cell characteristics. CLL is a disease of middle or old age. In CML, the characteristic feature is the predominance of granulocytic cells of all stages of differentiation in blood, bone marrow, liver, spleen, and other organs. In the symptomatic patient at diagnosis, the total white blood cell (WBC) count is usually about 200,000/μL, but may reach 1,000,000/μL. CML is relatively easy to diagnose because of the presence of the Philadelphia chromosome.

A “17p deletion” refers to deletion of the short (petit) arm of chromosome 17 in a CLL patient. A 17p deletion can indicate poor prognosis in a CLL patient.

The transcription factor p53, a tumor suppressor, also has an important role in the regulation of cellular metabolism. Id. The loss or mutation of p53 in tumor cells may be a significant contributor to changes in tumor cell metabolism to the glycolytic pathway. Id. The OCT1 transcription factor, another potential target for chemotherapeutics, may cooperate with p53 in regulating tumor cell metabolism. Id. Accordingly, a “p53 mutation” as used herein refers to any mutation of the p53 protein (including the gene encoding the protein) that modulates the activity of p53.

In addition to the acute and chronic categorization, neoplasms are also categorized based upon the cells giving rise to such disorder into precursor or peripheral. See e.g., U.S. patent publication no. 2008/0051379, the disclosure of which is incorporated herein by reference in its entirety. Precursor neoplasms include ALLs and lymphoblastic lymphomas and occur in lymphocytes before they have differentiated into either a T- or B-cell. Peripheral neoplasms are those that occur in lymphocytes that have differentiated into either T- or B-cells. Such peripheral neoplasms include, but are not limited to, B-cell CLL, B-cell prolymphocytic leukemia, lymphoplasmacytic lymphoma, mantle cell lymphoma, follicular lymphoma, extranodal marginal zone B-cell lymphoma of mucosa-associated lymphoid tissue, nodal marginal zone lymphoma, splenic marginal zone lymphoma, hairy cell leukemia, plasmacytoma, diffuse large B-cell lymphoma and Burkitt lymphoma. In over 95 percent of CLL cases, the clonal expansion is of a B cell lineage. See Cancer: Principles & Practice of Oncology (3rd Edition) (1989) (pp. 1843-1847). In less than 5 percent of CLL cases, the tumor cells have a T-cell phenotype. Notwithstanding these classifications, however, the pathological impairment of normal hematopoiesis is the hallmark of all leukemias.

Multiple myeloma (MM) is a cancer of plasma cells in the bone marrow. Normally, plasma cells produce antibodies and play a key role in immune function. However, uncontrolled growth of these cells leads to bone pain and fractures, anemia, infections, and other complications. Multiple myeloma is the second most common hematological malignancy, although the exact causes of multiple myeloma remain unknown. Multiple myeloma causes high levels of proteins in the blood, urine, and organs, including but not limited to M-protein and other immunoglobulins (antibodies), albumin, and beta-2-microglobulin. M-protein, short for monoclonal protein, also known as paraprotein, is a particularly abnormal protein produced by the myeloma plasma cells and can be found in the blood or urine of almost all patients with multiple myeloma.

Skeletal symptoms, including bone pain, are among the most clinically significant symptoms of multiple myeloma. Malignant plasma cells release osteoclast stimulating factors (including IL-1, IL-6 and TNF) that cause calcium to be leached from bones causing lytic lesions; hypercalcemia is another symptom. The osteoclast stimulating factors, also referred to as cytokines, may prevent apoptosis, or death of myeloma cells. Fifty percent of patients have radiologically detectable myeloma-related skeletal lesions at diagnosis. Other common clinical symptoms for multiple myeloma include polyneuropathy, anemia, hyperviscosity, infections, and renal insufficiency.

The term “relapsed” refers to a situation where a subject or a mammal that has had a remission of lymphoma or leukemia after therapy has a return of cancer cells.

As used herein, unless otherwise specified, the term “pharmaceutically acceptable salt(s),” includes, but is not limited to, salts of acidic or basic moieties of compounds described herein (e.g., Compound A). Basic moieties are capable of forming a wide variety of salts with various inorganic and organic acids. The acids that can be used to prepare pharmaceutically acceptable acid addition salts of such basic compounds are those that form non-toxic acid addition salts, e.g., salts containing pharmacologically acceptable anions. Suitable organic acids include, but are not limited to, maleic, fumaric, benzoic, ascorbic, succinic, acetic, formic, oxalic, propionic, tartaric, salicylic, citric, gluconic, lactic, mandelic, cinnamic, oleic, tannic, aspartic, stearic, palmitic, glycolic, glutamic, gluconic, glucaronic, saccharic, isonicotinic, methanesulfonic, ethanesulfonic, p-toluenesulfonic, benzenesulfonic acids, or pamoic (e.g., 1,1′-methylene-bis-(2-hydroxy-3-naphthoate)) acids. Suitable inorganic acids include, but are not limited to, hydrochloric, hydrobromic, hydroiodic, sulfuric, phosphoric, or nitric acids. Compounds that include an amine moiety can form pharmaceutically acceptable salts with various amino acids, in addition to the acids mentioned above. Chemical moieties that are acidic in nature are capable of forming base salts with various pharmacologically acceptable cations. Examples of such salts are alkali metal or alkaline earth metal salts and, particularly, calcium, magnesium, sodium, lithium, zinc, potassium, or iron salts.

As used herein, and unless otherwise specified, the term “solvate” means a compound that further includes a stoichiometric or non-stoichiometric amount of solvent bound by non-covalent intermolecular forces. Where the solvent is water, the solvate is a hydrate.

As used herein, and unless otherwise specified, the term “stereoisomer” encompasses all enantiomerically/stereomerically pure and enantiomerically/stereomerically enriched compounds provided herein.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, greater than about 98% by weight of one stereoisomer of the compound and less than about 2% by weight of the other stereoisomers of the compound or greater than about 99% by weight of one stereoisomer of the compound and less than about 1% by weight of the other stereoisomers of the compound.

As used herein and unless otherwise indicated, the term “stereomerically enriched” means a composition that comprises greater than about 55% by weight of one stereoisomer of a compound, greater than about 60% by weight of one stereoisomer of a compound, greater than about 70% by weight, or greater than about 80% by weight of one stereoisomer of a compound.

As used herein, and unless otherwise indicated, the term “enantiomerically pure” means a stereomerically pure composition of a compound having one chiral center. Similarly, the term “enantiomerically enriched” means a stereomerically enriched composition of a compound having one chiral center.

As used herein, and unless otherwise specified, the term “stable,” when used in connection with a formulation or a dosage form, means that the active ingredient of the formulation or dosage form remains solubilized for a specified amount of time and does not significantly degrade or aggregate or become otherwise modified (e.g., as determined, for example, by HPLC). In some embodiments, about 70 percent or greater, about 80 percent or greater or about 90 percent or greater of the compound remains solubilized after the specified period.

A “pharmaceutically acceptable excipient,” refers to a substance that aids the administration of an active agent to a subject by for example, modifying the stability of an active agent or modifying the absorption by a subject upon administration. A pharmaceutically acceptable excipient typically has no significant adverse toxicological effect on the patient. Examples of pharmaceutically acceptable excipients include, for example, water, NaCl (including salt solutions), normal saline solutions, sucrose, glucose, binders, fillers, disintegrants, lubricants, coatings, sweeteners, flavors, alcohols, oils, gelatins, carbohydrates such as amylose or starch, fatty acid esters, hydroxymethycellulose, polyvinyl pyrrolidine, and colors, and the like. One of skill in the art will recognize that other pharmaceutical excipients known in the art are useful in the present invention and include those listed in for example, the Handbook of Pharmaceutical Excipients, Rowe R. C., Shesky P. J., and Quinn M. E., 6^{th} Ed., The Pharmaceutical Press, RPS Publishing (2009). The terms “binder,” “filler,” “disintegrant,” and “lubricant” are used in accordance with the plain and ordinary meanings within the art.

An “anti-CD20 antibody” refers to an antibody specific for the CD20 antigen. Examples of known anti-CD20 antibodies include, but are not limited to, rituximab, ibritumomab (Zevaline®), tiuxetan, tositumomab, and ofatumumab (Arzerra®). Additional anti-CD20 antibody therapeutics that have been or are currently being developed are ocaratuzumab, ocrelizumab, veltuzumab (IMMU-106), AMR-133v, and TRU-015.

“Rituximab” refers to a chimeric CD20-directed cytolytic antibody. Rituximab therefore targets and kills B-cells and is useful in treating diseases characterized by B-cell dysfunction. Rituximab is marketed under the trade names Rituxan®, MabThera®, and Zytux®. Rituximab is known to deplete normal host B cells. See M. Aklilu et al., Annals of Oncology 15:1109-1114, 2004. The long-term immunologic effects of B cell depletion with rituximab and the characteristics of the reconstituting B cell pool in lymphoma patients are not well defined, despite the widespread usage of this therapy. See Jennifer H. Anolik et al., Clinical Immunology, vol. 122, issue 2, February 2007, pages 139-145. Rituximab is approved for use in treating lymphomas and leukemias including NHL and CLL.

“Obinutuzumab” refers to a humanized type II monoclonal antibody that binds to CD20 and triggers destruction of B-cells. Obinutuzumab is useful in treating diseases characterized by B-cell dysfunction. Obinutuzumab is marketed under the trade name Gazyva®. Obinutuzumab is approved for use in treating CLL.

“Ibrutinib” refers to an anti-cancer agent having the formula below:

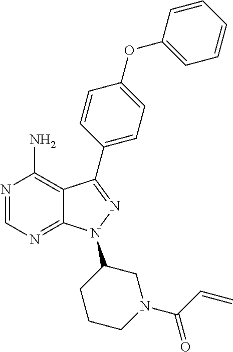

Ibrutinib as used herein also refers to enantiomers or a mixture of enantiomers thereof, pharmaceutically acceptable stereoisomers, pharmaceutically acceptable salts, prodrugs, solvates, hydrates, co-crystals, clathrates, or polymorphs thereof. In various embodiments, ibrutinib refers to the base, a pharmaceutically acceptable salt or solvate thereof.

Ibrutinib has specificity for B-cells and is useful in treating B-cell malignancies. Ibrutinib is marketed under the trade name Imbruvica®. Ibrutinib is approved for use in treating CLL and MCL.

As used herein, unless otherwise specified, the terms “about” and “approximately” mean an acceptable error for a particular value as determined by one of ordinary skill in the art, which depends in part on how the value is measured or determined. In certain embodiments, the term “about” and “approximately” mean within 1, 2, 3, or 4 standard deviations. In certain embodiments, the term “about” and “approximately” mean within 50%, 20%, 15%, 10%, 9%, 8%, 7%, 6%, 5%, 4%, 3%, 2%, 1%, 0.5%, or 0.05% of a given value or range.

As used herein, the term “biosimilar” (for example, of an approved reference product/biological drug, such as a protein therapeutic, antibody, etc.) refers to a biologic product that is similar to the reference product based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought (e.g., that there are no clinically meaningful differences between the biological product and the reference product in terms of the safety, purity, and potency of the product).

### Methods of Treating

The approach for patients with relapsed or refractory disease relies heavily on experimental treatments followed by stem cell transplantation, which may not be appropriate for patients with a poor performance status or advanced age. Therefore, a tremendous demand exists for new methods that can be used to treat patients with NHL.

The link between cancer and altered cellular metabolism has been well established. See Cairns, R. A., et al. Nature Rev., 2011, 11:85-95. Understanding tumor cell metabolism and the associated genetic changes thereof may lead to the identification of improved methods of cancer treatment. Id. For example, tumor cell survival and proliferation via increased glucose metabolism has been linked to the PIK3 pathway, whereby mutations in tumor suppressor genes such as PTEN activate tumor cell metabolism. Id. AKT1 (a.k.a., PKB) stimulates glucose metabolism associated with tumor cell growth by various interactions with PFKFB3, ENTPD5, mTOR and TSC2 (a.k.a., tuberin). Id.

Transcription factors HIF1 and HIF2 are largely responsible for cellular response to low oxygen conditions often associated with tumors. Id. Once activated, HIF1 promotes tumor cell capacity to carry out glycolysis. Id. Thus, inhibition of HIF1 may slow or reverse tumor cell metabolism. Activation of HIF1 has been linked to PI3K, tumor suppressor proteins such as VHL, succinate dehydrogenase (SDH) and fumarate hydratase. Id. The oncogenic transcription factor MYC has also been linked to tumor cell metabolism, specifically glycolysis. Id. MYC also promotes cell proliferation by glutamine metabolic pathways. Id.

AMP-activated protein kinase (AMPK) functions as a metabolic checkpoint that tumor cells must overcome in order to proliferate. Id. Several mutations have been identified that suppress AMPK signaling in tumor cells. See Shackelford, D. B. & Shaw, R. J., Nature Rev. Cancer, 2009, 9: 563-575. STK11 has been identified as a tumor suppressor gene related to the role of AMPK. See Cairns, R. A., et al. Nature Rev., 2011, 11:85-95.

Pyruvate kinate M2 (PKM2) promotes changes in cellular metabolism that confer metabolic advantages to cancer cells by supporting cell proliferation. Id. For example, lung cancer cells that express PKM2 over PKM1 have been found to have such an advantage. Id. In the clinic, PKM2 has been identified as being overexpressed in a number of cancer types. Id. Thus, PKM2 may be a useful biomarker for the early detection of tumors.

Mutations in isocitrate dehydrogenases IDH1 and IDH2 have been linked to tumorigenesis, specifically, in glioblastoma and acute myeloid leukemia. See Mardis, E. R. et al., N. Engl. J. Med., 2009, 361: 1058-1066; Parsons, D. W. et al., Science, 2008, 321: 1807-1812.

The incidence of cancer continues to climb as the general population ages, as new cancers develop, and as susceptible populations (e.g., people infected with AIDS, the elderly or excessively exposed to sunlight) grow. A tremendous demand therefore exists for new methods, treatments and compositions that can be used to treat patients with cancer including but not limited to those with lymphoma, NHL, multiple myeloma, AML, leukemias, and solid tumors.

Accordingly, compounds that can control and/or inhibit unwanted angiogenesis or inhibit the production of certain cytokines, including TNF-α, may be useful in the treatment and prevention of various forms of lymphoma and leukemia.

Provided herein are methods of treating, preventing, or managing (e.g., preventing the recurrence, or lengthening the time of remission of) cancer, including but not limited cancers involving solid tumors or blood borne tumors. Examples of types of solid tumors that may be treated, prevented or managed by various embodiments of the present invention include but are not limited to malignant melanoma, adrenal carcinoma, breast carcinoma, multiple myeloma, renal cell cancer, carcinoma of the pancreas, non-small-cell lung carcinoma (NSCLC) and carcinomas of unknown primary origin. The methods comprise administering to a patient in need of such treatment or prevention a therapeutically or prophylactically effective amount of Compound A:

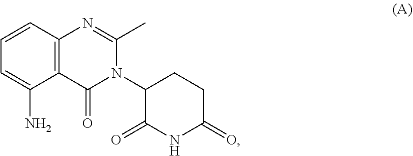 [SMILES: Cc1nc2cccc(N)c2c(=O)n1C1CCC(=O)NC1=O]

or a pharmaceutically acceptable salt, solvate, or stereoisomer thereof, in combination with an effective amount of: (i) an anti-CD20 antibody, e.g., rituximab; or (ii) ibrutinib.

Also provided herein are methods of treating, preventing, or managing cancer, including primary and metastatic cancer, as well as cancer that is refractory or resistant to conventional chemotherapy. The methods comprise administering to a patient in need of such treatment or prevention a therapeutically or prophylactically effective amount of Compound A:

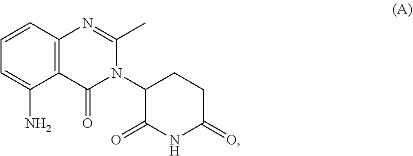 [SMILES: Cc1nc2cccc(N)c2c(=O)n1C1CCC(=O)NC1=O]

or a pharmaceutically acceptable salt, solvate, or stereoisomer thereof, in combination with an effective amount of: (i) an anti-CD20 antibody, e.g., rituximab; or (ii) ibrutinib.

Also provided herein are methods of treating, preventing, or managing lymphoma or leukemia by administering to a subject in need thereof an effective amount of Compound A:

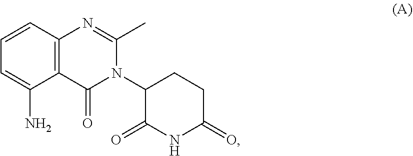 [SMILES: Cc1nc2cccc(N)c2c(=O)n1C1CCC(=O)NC1=O]

or a pharmaceutically acceptable salt, solvate, or stereoisomer thereof, in combination with an effective amount of (i) an anti-CD20 antibody, e.g., rituximab; or (ii) ibrutinib. In some embodiments, the lymphoma is selected from the group consisting of Hodgkin's lymphoma, non-Hodgkin's lymphoma (including but not limited to indolent non-Hodgkin's lymphoma (iNHL)), AIDS-related lymphomas, anaplastic large-cell lymphoma, angioimmunoblastic lymphoma, blastic NK-cell lymphoma, Burkitt's lymphoma, Burkitt-like lymphoma (small non-cleaved cell lymphoma, small lymphocytic lymphoma, cutaneous T-cell lymphoma, diffuse large B-cell lymphoma, enteropathy-type T-cell lymphoma, lymphoblastic lymphoma, mantle cell lymphoma, marginal zone lymphoma, nasal T-cell lymphoma, pediatric lymphoma, peripheral T-cell lymphoma, primary central nervous system lymphoma, transformed lymphoma, treatment-related T-cell lymphoma and Waldenstrom's macroglobulinemia. In certain embodiments, the leukemia is selected from the group consisting of acute myeloid leukemia (AML), T-cell leukemia, chronic myeloid leukemia (CML), chronic lymphocytic leukemia (CLL) and acute lymphoblastic leukemia (ALL).

In one aspect is a method of treating lymphoma by administering to a subject in need thereof. Compound A as described herein in combination with an anti-CD20 antibody as described herein.

In another aspect is a method of preventing lymphoma by administering to a subject in need thereof. Compound A as described herein in combination with an anti-CD20 antibody as described herein.

In yet another aspect is a method of managing lymphoma by administering to a subject in need thereof. Compound A as described herein in combination with an anti-CD20 antibody as described herein.

In still another aspect is a method of treating leukemia by administering to a subject in need thereof. Compound A as described herein in combination with an anti-CD20 antibody as described herein.

In another aspect is a method of preventing leukemia by administering to a subject in need thereof. Compound A as described herein in combination with an anti-CD20 antibody as described herein.

In yet another aspect is a method of managing leukemia by administering to a subject in need thereof. Compound A as described herein in combination with an anti-CD20 antibody as described herein.

Compound A can be prepared according to the methods described in the Examples provided herein or as described in U.S. Pat. No. 7,635,700, the disclosure of which is incorporated herein by reference in its entirety. The compound can also be synthesized according to other methods apparent to those of skill in the art based upon the teaching herein.

Compound A markedly inhibits TNF-α, IL-1β, and other inflammatory cytokines in LPS-stimulated hPBMC and human whole blood. TNF-α is an inflammatory cytokine produced by macrophages and monocytes during acute inflammation. TNF-α is responsible for a diverse range of signaling events within cells. TNF-α may play a pathological role in cancer. Without being limited by theory, one of the biological effects exerted by Compound A is the reduction of synthesis of TNF-α. Compound A enhances the degradation of TNF-α mRNA. Compound A also potently inhibits IL-1 β and stimulates IL-10 under these conditions.

Further, without being limited by theory, Compound A is a potent co-stimulator of T cells and increased cell proliferation in a dose dependent manner under appropriate conditions.

In certain embodiments, without being limited by theory, the biological effects exerted by Compound A include, but are not limited to, anti-angiogenic and immune modulating effects.

In certain embodiments, Compound A is a solid. In certain embodiments, Compound A is hydrated. In certain embodiments, Compound A is solvated. In certain embodiments, Compound A is anhydrous. In certain embodiments, Compound A is nonhygroscopic.

In certain embodiments, Compound A is amorphous. In certain embodiments, Compound A is crystalline. In certain embodiments, Compound A is in a crystalline form described in U.S. Publication No. 2012/0232100-A1, which is incorporated herein by reference in its entirety.

The solid forms of Compound A can be prepared according to the methods described in the disclosure of U.S. Publication No. 2012/0232100-A1. The solid forms can also be prepared according to other methods apparent to those of skill in the art.

In certain embodiments, Compound A is a hydrochloride salt of 3-(5-amino-2-methyl-4-oxo-4H-quinazolin-3-yl)-piperidine-2,6-dione, or an enantiomer or a mixture of enantiomers thereof; or a pharmaceutically acceptable solvate, hydrate, co-crystal, clathrate, or polymorph thereof. In certain embodiments, the hydrochloride salt is a solid. In certain embodiments, the hydrochloride salt is anhydrous. In certain embodiments, the hydrochloride salt is nonhygroscopic. In certain embodiments, the hydrochloride salt is amorphous. In certain embodiments, the hydrochloride salt is crystalline. In certain embodiments, the hydrochloride salt is in crystalline Form A.

The hydrochloride salt of Compound A and solid forms thereof can be prepared according to the methods described in the disclosure of U.S. Publication No. 2012/0232100-A1. The hydrochloride salt and the solid forms thereof can be also prepared according to other methods apparent to those of skill in the art.

Compound A provided herein contains one chiral center, and can exist as a mixture of enantiomers, e.g., a racemic mixture. This disclosure encompasses the use of stereomerically pure forms of such a compound, as well as the use of mixtures of those forms. For example, mixtures comprising equal or unequal amounts of the enantiomers of Compound A provided herein may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

In some embodiments, Compound A can contain unnatural proportions of atomic isotopes at one or more of the atoms. For example, the compound may be radiolabeled with radioactive isotopes, such as for example, tritium (3H), iodine-125 (1251), sulfur 35 (35S), or carbon-14 (14C), or may be isotopically enriched, such as with deuterium (2H), carbon-13 (13C), or nitrogen-15 (15N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, research reagents, e.g., binding assay reagents, and diagnostic agents, e.g., in vivo imaging agents. All isotopic variations of Compound A, whether radioactive or not, are intended to be encompassed within the scope of the embodiments provided herein. In some embodiments, provided herein are isotopologues of Compound A, for example, the isotopologues are deuterium, carbon-13, or nitrogen-15 enriched Compound A.

In certain embodiment, Compound A, or a pharmaceutically acceptable salt, solvate, or stereoisomer thereof, is administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, Compound A, or a pharmaceutically acceptable salt, solvate, or stereoisomer thereof, is orally administered.

Compound A, or a pharmaceutically acceptable salt, solvate, or stereoisomer thereof, is administered at an amount of about 0.01 mg to about 100 mg per day, about 0.1 mg to about 75 mg per day, about 0.5 mg to about 50 mg per day, about 0.1 mg to about 25 mg per day, about 1 mg to about 25 mg per day, about 0.25 mg to about 10 mg per day, about 0.5 mg to about 10 mg per day, about 1 mg to about 10 mg per day, about 0.25 mg to about 5 mg per day, about 0.5 mg to about 5 mg per day, about 1 mg to about 5 mg per day, about 1 mg to about 3 mg per day, or about 2 mg to about 5 mg per day.

In one embodiment, Compound A, or a pharmaceutically acceptable salt, solvate, or stereoisomer thereof, is administered at an amount of about 0.25 mg to about 5 mg per day. In one embodiment, Compound A, or a pharmaceutically acceptable salt, solvate, or stereoisomer thereof, is administered at an amount of about 1 mg to about 5 mg per day. In one embodiment, Compound A, or a pharmaceutically acceptable salt, solvate, or stereoisomer thereof, is administered at an amount of about 0.25 mg per day. In one embodiment, Compound A, or a pharmaceutically acceptable salt, solvate, or stereoisomer thereof, is administered at an amount of about 0.5 mg per day. In one embodiment, Compound A, or a pharmaceutically acceptable salt, solvate, or stereoisomer thereof, is administered at an amount of about 1 mg per day. In one embodiment, Compound A, or a pharmaceutically acceptable salt, solvate, or stereoisomer thereof, is administered at an amount of about 2 mg per day. In one embodiment, Compound A, or a pharmaceutically acceptable salt, solvate, or stereoisomer thereof, is administered at an amount of about 2.5 mg per day. In one embodiment, Compound A, or a pharmaceutically acceptable salt, solvate, or stereoisomer thereof, is administered at an amount of about 3 mg per day. In one embodiment, Compound A, or a pharmaceutically acceptable salt or solvate thereof, is administered at an amount of about 4 mg per day.

In one embodiment, Compound A, or a pharmaceutically acceptable salt, solvate, or stereoisomer thereof, is administered once daily for 28 consecutive days in a 28 days cycle. In one embodiment, Compound A, or a pharmaceutically acceptable salt, solvate, or stereoisomer thereof, is administered once daily for 5 consecutive days followed by 2 days of no administration in a 28 days cycle. In one embodiment, Compound A, or a pharmaceutically acceptable salt, solvate, or stereoisomer thereof, is administered once daily for 21 consecutive days followed by 7 days of no administration in a 28 days cycle.

In certain embodiment, an anti-CD20 antibody described herein or ibrutinib, or a pharmaceutically acceptable salt or solvate thereof, is administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, ibrutinib, or a pharmaceutically acceptable salt or solvate thereof, is orally administered. In another embodiment, an anti-CD20 antibody is administered intravenously.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure.

The anti-CD20 antibody can be rituximab or obinutuzumab. In certain embodiments, the anti-CD20 antibody administered is rituximab marketed as Rituxan®. When Compound A, or a pharmaceutically acceptable salt, solvate, or stereoisomer thereof, is co-administered with rituximab, rituximab can be administered at a concentration of 375 mg/m². In such embodiments, Compound A, or a pharmaceutically acceptable salt, solvate, or stereoisomer thereof, can be administered daily at a concentration described herein. Compound A, or a pharmaceutically acceptable salt, solvate, or stereoisomer thereof, can be administered cyclically as described herein in combination with rituximab.

In one embodiment, rituximab is administered on days 1 and 8 of cycle 1 (e.g., the first cycle) at a concentration of 375 mg/m². Rituximab can further be administered on day one of every additional cycle in a 28-day cycle at a concentration of 500 mg/m². In such embodiments, rituximab can be administered over 1, 2, 3, 4, 5, 6, 7, 8, 9, 10, or 11 cycles.

In one embodiment, rituximab is administered in combination with Compound A, or a pharmaceutically acceptable salt, solvate, or stereoisomer thereof, over a maximum of 11 cycles.

In one embodiment, Compound A, or a pharmaceutically acceptable salt, solvate, or stereoisomer thereof, is administered once daily for 5 consecutive days followed by 2 days of no administration in a 28 days cycle, and rituximab, is administered on day one of the first cycle at a concentration of 375 mg/m² and at a concentration of 500 mg/m² on the first day of each cycle thereafter.

In another embodiment, Compound A, or a pharmaceutically acceptable salt, solvate, or stereoisomer thereof, is administered once daily for 21 consecutive days followed by 7 days of no administration in a 28 days cycle, and rituximab, is administered on day one of the first cycle at a concentration of 375 mg/m² and at a concentration of 500 mg/m² on the first day of each cycle thereafter.

In one embodiment, Compound A, or a pharmaceutically acceptable salt, solvate, or stereoisomer thereof, is orally administered at an amount of about 0.25 mg to about 5 mg per day and rituximab, is administered on day one of the first cycle at a concentration of 375 mg/m² and at a concentration of 500 mg/m² on the first day of each cycle thereafter.

In one embodiment, Compound A, or a pharmaceutically acceptable salt, solvate, or stereoisomer thereof, is orally administered at an amount of about 0.25, 0.5, 1, 2, 2.5, 3, 4, or 5 mg per day and rituximab, is administered on day one of the first cycle at a concentration of 375 mg/m² and at a concentration of 500 mg/m² on the first day of each cycle thereafter.

In one embodiment, Compound A, or a pharmaceutically acceptable salt, solvate, or stereoisomer thereof, is orally administered at an amount of about 0.25 mg per day and rituximab, is administered on day one of the first cycle at a concentration of 375 mg/m² and at a concentration of 500 mg/m² on the first day of each cycle thereafter.

In one embodiment, Compound A, or a pharmaceutically acceptable salt, solvate, or stereoisomer thereof, is orally administered at an amount of about 0.5 mg per day and rituximab, is administered on day one of the first cycle at a concentration of 375 mg/m² and at a concentration of 500 mg/m² on the first day of each cycle thereafter.

In one embodiment, Compound A, or a pharmaceutically acceptable salt, solvate, or stereoisomer thereof, is orally administered at an amount of about 1 mg per day and rituximab, is administered on day one of the first cycle at a concentration of 375 mg/m² and at a concentration of 500 mg/m² on the first day of each cycle thereafter.

In one embodiment, Compound A, or a pharmaceutically acceptable salt, solvate, or stereoisomer thereof, is orally administered at an amount of about 2 mg per day and rituximab, is administered on day one of the first cycle at a concentration of 375 mg/m² and at a concentration of 500 mg/m² on the first day of each cycle thereafter.

In one embodiment, Compound A, or a pharmaceutically acceptable salt, solvate, or stereoisomer thereof, is orally administered at an amount of about 2.5 mg per day and rituximab, is administered on day one of the first cycle at a concentration of 375 mg/m² and at a concentration of 500 mg/m² on the first day of each cycle thereafter.

In one embodiment, Compound A, or a pharmaceutically acceptable salt, solvate, or stereoisomer thereof, is orally administered at an amount of about 3 mg per day and rituximab, is administered on day one of the first cycle at a concentration of 375 mg/m² and at a concentration of 500 mg/m² on the first day of each cycle thereafter.

In one embodiment, Compound A, or a pharmaceutically acceptable salt, solvate, or stereoisomer thereof, is orally administered at an amount of about 4 mg per day and rituximab, is administered on day one of the first cycle at a concentration of 375 mg/m² and at a concentration of 500 mg/m² on the first day of each cycle thereafter.

In one embodiment, Compound A, or a pharmaceutically acceptable salt, solvate, or stereoisomer thereof, is orally administered at an amount of about 5 mg per day and rituximab, is administered on day one of the first cycle at a concentration of 375 mg/m² and at a concentration of 500 mg/m² on the first day of each cycle thereafter.

When Compound A, or a pharmaceutically acceptable salt, solvate, or stereoisomer thereof, and rituximab are co-administered, the subject may be a subject in need thereof who was not previously treated with an anti-CD20 antibody (e.g., rituximab or obinutuzumab). When Compound A, or a pharmaceutically acceptable salt, solvate, or stereoisomer thereof, and rituximab are co-administered, the subject may be a subject in need thereof who was not previously treated with ibrutinib.

When Compound A, or a pharmaceutically acceptable salt, solvate, or stereoisomer thereof, and rituximab are co-administered, the subject may be a subject in need thereof who was previously treated with an anti-CD20 antibody (e.g., rituximab or obinutuzumab). When Compound A, or a pharmaceutically acceptable salt, solvate, or stereoisomer thereof, and rituximab are co-administered, the subject may be a subject in need thereof who was previously treated with ibrutinib. In some embodiments, when Compound A, or a pharmaceutically acceptable salt, solvate, or stereoisomer thereof, and rituximab are co-administered, the subject has CLL having a p17 deletion and/or p53 mutation. In another embodiment, when Compound A, or a pharmaceutically acceptable salt, solvate, or stereoisomer thereof, and rituximab are co-administered the subject has CLL having a p17 deletion and/or p53 mutation and has been previously treated with at least one other cancer therapy. In some embodiments the at least one other cancer therapy is prior treatment with ibrutinib.

The anti-CD20 antibody can be obinutuzumab. In certain embodiments, the anti-CD20 antibody administered is obinutuzumab marketed as Gazyva. When Compound A, or a pharmaceutically acceptable salt, solvate, or stereoisomer thereof, is co-administered with obinutuzumab, obinutuzumab can be administered at an amount of 100 mg on day one of cycle 1, 900 mg on day 2 of cycle 1, and 1000 mg on days 8 and 15 of cycle 1. Obinutuzumab can be further administered in combination with Compound A, or a pharmaceutically acceptable salt, solvate, or stereoisomer thereof, on day one of every cycle in a 28-day cycle at an amount of 1000 mg. In such embodiments, obinutuzumab can be administered over 1, 2, 3, 4, 5, or 6 cycles. In such embodiments, Compound A, or a pharmaceutically acceptable salt, solvate, or stereoisomer thereof, can be administered daily at a concentration described herein. Compound A, or a pharmaceutically acceptable salt, solvate, or stereoisomer thereof, can be administered cyclically as described herein in combination with obinutuzumab.

In one embodiment, obinutuzumab is administered in combination with Compound A, or a pharmaceutically acceptable salt, solvate, or stereoisomer thereof, over a maximum of 6 cycles.

In one embodiment, Compound A, or a pharmaceutically acceptable salt, solvate, or stereoisomer thereof, is administered once daily for 5 consecutive days followed by 2 days of no administration in a 28 days cycle, and obinutuzumab is administered at an amount of 100 mg on day one of cycle 1, 900 mg on day 2 of cycle 1, and 1000 mg on days 8 and 15 of cycle 1. Obinutuzumab can be further administered in such embodiments at an amount of 1000 mg on day 1 of each cycle thereafter.

In another embodiment, Compound A, or a pharmaceutically acceptable salt, solvate, or stereoisomer thereof, is administered once daily for 21 consecutive days followed by 7 days of no administration in a 28 days cycle, and obinutuzumab is administered at an amount of 100 mg on day one of cycle 1, 900 mg on day 2 of cycle 1, and 1000 mg on days 8 and 15 of cycle 1. Obinutuzumab can be further administered in such embodiments at an amount of 1000 mg on day 1 of each cycle thereafter.

In one embodiment, Compound A, or a pharmaceutically acceptable salt, solvate, or stereoisomer thereof, is orally administered at an amount of about 0.25 mg to about 5 mg per day and obinutuzumab is administered at an amount of 100 mg on day one of cycle 1, 900 mg on day 2 of cycle 1, and 1000 mg on days 8 and 15 of cycle 1. Obinutuzumab can be further administered in such embodiments at an amount of 1000 mg on day 1 of each cycle thereafter.

In one embodiment, Compound A, or a pharmaceutically acceptable salt, solvate, or stereoisomer thereof, is orally administered at an amount of about 0.25, 0.5, 1, 2, 2.5, 3, 4, or 5 mg per day and obinutuzumab is administered at an amount of 100 mg on day one of cycle 1, 900 mg on day 2 of cycle 1, and 1000 mg on days 8 and 15 of cycle 1. Obinutuzumab can be further administered in such embodiments at an amount of 1000 mg on day 1 of each cycle thereafter.

In one embodiment, Compound A, or a pharmaceutically acceptable salt, solvate, or stereoisomer thereof, is orally administered at an amount of about 0.25 mg per day and obinutuzumab is administered at an amount of 100 mg on day one of cycle 1, 900 mg on day 2 of cycle 1, and 1000 mg on days 8 and 15 of cycle 1. Obinutuzumab can be further administered in such embodiments at an amount of 1000 mg on day 1 of each cycle thereafter.

In one embodiment, Compound A, or a pharmaceutically acceptable salt, solvate, or stereoisomer thereof, is orally administered at an amount of about 0.5 mg per day and obinutuzumab is administered at an amount of 100 mg on day one of cycle 1, 900 mg on day 2 of cycle 1, and 1000 mg on days 8 and 15 of cycle 1. Obinutuzumab can be further administered in such embodiments at an amount of 1000 mg on day 1 of each cycle thereafter.

In one embodiment, Compound A, or a pharmaceutically acceptable salt, solvate, or stereoisomer thereof, is orally administered at an amount of about 1 mg per day and obinutuzumab is administered at an amount of 100 mg on day one of cycle 1, 900 mg on day 2 of cycle 1, and 1000 mg on days 8 and 15 of cycle 1. Obinutuzumab can be further administered in such embodiments at an amount of 1000 mg on day 1 of each cycle thereafter.

In one embodiment, Compound A, or a pharmaceutically acceptable salt, solvate, or stereoisomer thereof, is orally administered at an amount of about 2 mg per day and obinutuzumab is administered at an amount of 100 mg on day one of cycle 1, 900 mg on day 2 of cycle 1, and 1000 mg on days 8 and 15 of cycle 1. Obinutuzumab can be further administered in such embodiments at an amount of 1000 mg on day 1 of each cycle thereafter.

In one embodiment, Compound A, or a pharmaceutically acceptable salt, solvate, or stereoisomer thereof, is orally administered at an amount of about 2.5 mg per day and obinutuzumab is administered at an amount of 100 mg on day one of cycle 1, 900 mg on day 2 of cycle 1, and 1000 mg on days 8 and 15 of cycle 1. Obinutuzumab can be further administered in such embodiments at an amount of 1000 mg on day 1 of each cycle thereafter.

In one embodiment, Compound A, or a pharmaceutically acceptable salt, solvate, or stereoisomer thereof, is orally administered at an amount of about 3 mg per day and obinutuzumab is administered at an amount of 100 mg on day one of cycle 1, 900 mg on day 2 of cycle 1, and 1000 mg on days 8 and 15 of cycle 1. Obinutuzumab can be further administered in such embodiments at an amount of 1000 mg on day 1 of each cycle thereafter.

In one embodiment, Compound A, or a pharmaceutically acceptable salt, solvate, or stereoisomer thereof, is orally administered at an amount of about 4 mg per day and obinutuzumab is administered at an amount of 100 mg on day one of cycle 1, 900 mg on day 2 of cycle 1, and 1000 mg on days 8 and 15 of cycle 1. Obinutuzumab can be further administered in such embodiments at an amount of 1000 mg on day 1 of each cycle thereafter.

In one embodiment, Compound A, or a pharmaceutically acceptable salt, solvate, or stereoisomer thereof, is orally administered at an amount of about 5 mg per day and obinutuzumab is administered at an amount of 100 mg on day one of cycle 1, 900 mg on day 2 of cycle 1, and 1000 mg on days 8 and 15 of cycle 1. Obinutuzumab can be further administered in such embodiments at an amount of 1000 mg on day 1 of each cycle thereafter.

When Compound A, or a pharmaceutically acceptable salt, solvate, or stereoisomer thereof, and obinutuzumab are co-administered, the subject may be a subject in need thereof who was not previously treated with an anti-CD20 antibody (e.g., rituximab or obinutuzumab). When Compound A, or a pharmaceutically acceptable salt, solvate, or stereoisomer thereof, and obinutuzumab are co-administered, the subject may be a subject in need thereof who was not previously treated with ibrutinib.

When Compound A, or a pharmaceutically acceptable salt, solvate, or stereoisomer thereof, and obinutuzumab are co-administered, the subject may be a subject in need thereof who was previously treated with an anti-CD20 antibody (e.g., rituximab or obinutuzumab). When Compound A, or a pharmaceutically acceptable salt, solvate, or stereoisomer thereof, and obinutuzumab are co-administered, the subject may be a subject in need thereof who was previously treated with ibrutinib. In some embodiments, when Compound A, or a pharmaceutically acceptable salt, solvate, or stereoisomer thereof, and obinutuzumab are co-administered, the subject has CLL having a p17 deletion and/or p53 mutation. In another embodiment, when Compound A, or a pharmaceutically acceptable salt, solvate, or stereoisomer thereof, and obinutuzumab are co-administered the subject has CLL having a p17 deletion and/or p53 mutation and has been previously treated with at least one other cancer therapy. In some embodiments the at least one other cancer therapy is treatment with ibrutinib.

Further provided herein are methods of treating, preventing, or managing lymphoma or leukemia, said method comprising administering to a subject in need thereof. Compound A:

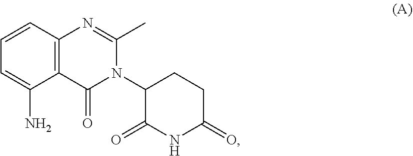 [SMILES: Cc1nc2cccc(N)c2c(=O)n1C1CCC(=O)NC1=O]

or a pharmaceutically acceptable salt, solvate, or stereoisomer thereof, in combination with ibrutinib, or a pharmaceutically acceptable salt or solvate thereof.

In one aspect is a method of treating lymphoma by administering to a subject in need thereof. Compound A as described herein in combination with ibrutinib as described herein. In another aspect is a method of preventing lymphoma by administering to a subject in need thereof. Compound A as described herein in combination with ibrutinib as described herein. In yet another aspect is a method of managing lymphoma by administering to a subject in need thereof. Compound A as described herein in combination with ibrutinib as described herein. In still another aspect is a method of treating leukemia by administering to a subject in need thereof. Compound A as described herein in combination with ibrutinib as described herein. In another aspect is a method of preventing leukemia by administering to a subject in need thereof. Compound A as described herein in combination with ibrutinib as described herein. In yet another aspect is a method of managing leukemia by administering to a subject in need thereof. Compound A as described herein in combination with ibrutinib as described herein.

When Compound A, or a pharmaceutically acceptable salt, solvate, or stereoisomer thereof, is co-administered with ibrutinib, ibrutinib can be administered at an amount of 420 mg. When Compound A, or a pharmaceutically acceptable salt, solvate, or stereoisomer thereof, is co-administered with ibrutinib, ibrutinib can be administered daily at an amount of 420 mg. In such embodiments, Compound A, or a pharmaceutically acceptable salt, solvate, or stereoisomer thereof, can be administered daily at a concentration described herein. Compound A, or a pharmaceutically acceptable salt, solvate, or stereoisomer thereof, can be administered cyclically as described herein in combination with ibrutinib.

In one embodiment, ibrutinib, or a pharmaceutically acceptable salt or solvate thereof is administered in combination with Compound A, or a pharmaceutically acceptable salt, solvate, or stereoisomer thereof, until disease progression or unacceptable toxicity develops.

In one embodiment, Compound A, or a pharmaceutically acceptable salt, solvate, or stereoisomer thereof, is administered once daily for 5 consecutive days followed by 2 days of no administration in a 28 days cycle, and ibrutinib, or a pharmaceutically acceptable salt or solvate thereof is administered daily at an amount of 420 mg.

In another embodiment, Compound A, or a pharmaceutically acceptable salt, solvate, or stereoisomer thereof, is administered once daily for 21 consecutive days followed by 7 days of no administration in a 28 days cycle, and ibrutinib, or a pharmaceutically acceptable salt or solvate thereof is administered daily at an amount of 420 mg.

In one embodiment, Compound A, or a pharmaceutically acceptable salt, solvate, or stereoisomer thereof, is orally administered at an amount of about 0.25 mg to about 5 mg per day and ibrutinib, or a pharmaceutically acceptable salt or solvate thereof is administered daily at an amount of 420 mg.

In one embodiment, Compound A, or a pharmaceutically acceptable salt, solvate, or stereoisomer thereof, is orally administered at an amount of about 0.25, 0.5, 1, 2, 2.5, 3, 4, or 5 mg per day and ibrutinib, or a pharmaceutically acceptable salt or solvate thereof is administered daily at an amount of 420 mg.

In one embodiment, Compound A, or a pharmaceutically acceptable salt, solvate, or stereoisomer thereof, is orally administered at an amount of about 0.25 mg per day and ibrutinib, or a pharmaceutically acceptable salt or solvate thereof is administered daily at an amount of 420 mg.

In one embodiment, Compound A, or a pharmaceutically acceptable salt, solvate, or stereoisomer thereof, is orally administered at an amount of about 0.5 mg per day and ibrutinib, or a pharmaceutically acceptable salt or solvate thereof is administered daily at an amount of 420 mg.

In one embodiment, Compound A, or a pharmaceutically acceptable salt, solvate, or stereoisomer thereof, is orally administered at an amount of about 1 mg per day and ibrutinib, or a pharmaceutically acceptable salt or solvate thereof is administered daily at an amount of 420 mg.

In one embodiment, Compound A, or a pharmaceutically acceptable salt, solvate, or stereoisomer thereof, is orally administered at an amount of about 2 mg per day and ibrutinib, or a pharmaceutically acceptable salt or solvate thereof is administered daily at an amount of 420 mg.

In one embodiment, Compound A, or a pharmaceutically acceptable salt, solvate, or stereoisomer thereof, is orally administered at an amount of about 2.5 mg per day and ibrutinib, or a pharmaceutically acceptable salt or solvate thereof is administered daily at an amount of 420 mg.

In one embodiment, Compound A, or a pharmaceutically acceptable salt, solvate, or stereoisomer thereof, is orally administered at an amount of about 3 mg per day and ibrutinib, or a pharmaceutically acceptable salt or solvate thereof is administered daily at an amount of 420 mg.

In one embodiment, Compound A, or a pharmaceutically acceptable salt, solvate, or stereoisomer thereof, is orally administered at an amount of about 4 mg per day and ibrutinib, or a pharmaceutically acceptable salt or solvate thereof is administered daily at an amount of 420 mg.

In one embodiment, Compound A, or a pharmaceutically acceptable salt, solvate, or stereoisomer thereof, is orally administered at an amount of about 5 mg per day and ibrutinib, or a pharmaceutically acceptable salt or solvate thereof is administered daily at an amount of 420 mg.

When Compound A, or a pharmaceutically acceptable salt, solvate, or stereoisomer thereof, and ibrutinib, or a pharmaceutically acceptable salt or solvate thereof are co-administered, the subject may be a subject in need thereof who was not previously treated with an anti-CD20 antibody (e.g., rituximab or obinutuzumab). When Compound A, or a pharmaceutically acceptable salt, solvate, or stereoisomer thereof, and ibrutinib, or a pharmaceutically acceptable salt or solvate thereof are co-administered, the subject may be a subject in need thereof who was not previously treated with ibrutinib, or a pharmaceutically acceptable salt or solvate thereof.

When Compound A, or a pharmaceutically acceptable salt, solvate, or stereoisomer thereof, and ibrutinib, or a pharmaceutically acceptable salt or solvate thereof are co-administered, the subject may be a subject in need thereof who was previously treated with an anti-CD20 antibody (e.g., rituximab or obinutuzumab). When Compound A, or a pharmaceutically acceptable salt, solvate, or stereoisomer thereof, and ibrutinib, or a pharmaceutically acceptable salt or solvate thereof are co-administered, the subject may be a subject in need thereof who was not previously treated with ibrutinib, or a pharmaceutically acceptable salt or solvate thereof. In some embodiments, when Compound A, or a pharmaceutically acceptable salt, solvate, or stereoisomer thereof, and ibrutinib, or a pharmaceutically acceptable salt or solvate thereof are co-administered, the subject has CLL having a p17 deletion and/or p53 mutation. In another embodiment, when Compound A, or a pharmaceutically acceptable salt, solvate, or stereoisomer thereof, and ibrutinib, or a pharmaceutically acceptable salt or solvate thereof are co-administered, the subject has CLL having a p17 deletion and/or p53 mutation and has been previously treated with at least one other cancer therapy. In some embodiments the at least one other cancer therapy is treatment with ibrutinib, or a pharmaceutically acceptable salt or solvate thereof, while in other embodiments, the at least one other cancer therapy is not ibrutinib.

Compound A, or a pharmaceutically acceptable salt, solvate, or stereoisomer thereof, and rituximab, obinutuzumab, or ibrutinib, or a pharmaceutically acceptable salt or solvate thereof, may be administered using the same route or via different routes. Accordingly, in certain embodiments, Compound A, or a pharmaceutically acceptable salt, solvate, or stereoisomer thereof can be administered orally and rituximab and obinutuzumab are administered intravenously (e.g., intravenous infusion). Compound A, or a pharmaceutically acceptable salt, solvate, or stereoisomer thereof can be administered orally and ibrutinib, or a pharmaceutically acceptable salt or solvate thereof can be administered orally.

Compound A, or a pharmaceutically acceptable salt, solvate, or stereoisomer thereof, and rituximab, obinutuzumab, or ibrutinib, or a pharmaceutically acceptable salt or solvate thereof, may be administered simultaneously or sequentially. Compound A, or a pharmaceutically acceptable salt, solvate, or stereoisomer thereof, and rituximab, obinutuzumab, or ibrutinib, or a pharmaceutically acceptable salt or solvate thereof, may be administered in one pharmaceutical composition or in separate compositions.

In certain embodiments, compounds provided herein can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles as described herein (i.e., including days, weeks, or months of rest without the drug or drugs).

In certain embodiments, Compound A, or a pharmaceutically acceptable salt, solvate, or stereoisomer thereof, and an anti-CD20 antibody (e.g., rituximab or obinutuzumab) or ibrutinib, or a pharmaceutically acceptable salt or solvate thereof, may be administered in combination with one or more additional active agents.

Examples of such additional agents include, but are not limited to: Abraxane®; ace-11; acivicin; aclarubicin; acodazole hydrochloride; acronine; adozelesin; aldesleukin; altretamine; ambomycin; ametantrone acetate; amrubicin; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; herceptin; hydroxyurea; idarubicin hydrochloride; ifosfamide; ilmofosine; iproplatin; irinotecan; irinotecan hydrochloride; lanreotide acetate; lapatinib; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; romidepsin; safingol; safingol hydrochloride; semustine; simtrazene; sparfosate sodium; sparsomycin; spirogermanium hydrochloride; spiromustine; spiroplatin; stem cell treatments such as PDA-001; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; venetoclax (ABT-199), verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; and immunotherapeutic agents such as PD1/PDL1, CD40/CD40L, and CD47d targeted agents that allow for the use of immunotherapy.

Other examples include, but are not limited to: 20 epi 1,25 dihydroxyvitamin D3; 5 ethynyluracil; abiraterone; aclarubicin; acylfulvene; adecypenol; adozelesin; aldesleukin; ALL TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti dorsalizing morphogenetic protein 1; antiandrogen, prostatic carcinoma; antiestrogen; antineoplaston; antisense oligonucleotides; aphidicolin glycinate; apoptosis gene modulators; apoptosis regulators; apurinic acid; ara CDP DL PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta alethine; betaclamycin B; betulinic acid; b FGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide amino triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro 5 azacytidine; dihydrotaxol, 9; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin like growth factor 1 receptor inhibitor; interferon agonists; interferons; interleukins; iobenguane; iododoxorubicin; ipomeanol, 4; iroplact; irsogladine; isobengazole; isohomohalicondrin B; itasetron; jasplakinolide; kahalalide F; lamellarin N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N acetyldinaline; N substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (GENASENSE®); 06 benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum triamine complex; porfimer sodium; porfiromycin; prednisone; propyl bis acridone; prostaglandin J2; proteasome inhibitors; protein A based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Accordingly, in such embodiments, Compound A, or a pharmaceutically acceptable salt, solvate, or stereoisomer thereof is co-administered as a combination therapy with at least two additional active agents, where one agent is rituximab, obinutuzumab, or ibrutinib, or a pharmaceutically acceptable salt or solvate thereof.

### Pharmaceutical Compositions

Provided herein are pharmaceutical compositions and dosage forms, which include compounds described herein. Thus, in one aspect is a pharmaceutical composition or dosage form that includes Compound A, or an enantiomer or a mixture of enantiomers thereof, or a pharmaceutically acceptable salt, solvate, hydrate, co-crystal, clathrate, or polymorph thereof and rituximab. In another aspect is a pharmaceutical composition or dosage form that includes Compound A, or an enantiomer or a mixture of enantiomers thereof, or a pharmaceutically acceptable salt, solvate, hydrate, co-crystal, clathrate, or polymorph thereof and obinutuzumab. In yet another aspect is a pharmaceutical composition or dosage form that includes Compound A, or an enantiomer or a mixture of enantiomers thereof, or a pharmaceutically acceptable salt, solvate, hydrate, co-crystal, clathrate, or polymorph thereof and ibrutinib, or a pharmaceutically acceptable salt or solvate thereof or a mixture thereof. The pharmaceutical compositions and dosage forms described herein can further include one or more pharmaceutically acceptable excipients.

In certain embodiments, pharmaceutical compositions and dosage forms provided herein also include one or more additional active ingredients. Examples of optional second, or additional, active ingredients are disclosed elsewhere herein.

Compounds described herein, and anti-cancer agents described herein (e.g., rituximab, obinutuzumab, ibrutinib, or a pharmaceutically acceptable salt or solvate thereof, or a second active agent described herein) can be provided in single unit dosage form. Single unit dosage forms provided herein are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal, or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms provided herein may vary depending on their use. For example, a dosage form used in the acute treatment of a disease may contain larger amounts of one or more of the active ingredients than a dosage form used in the chronic treatment of the same disease. Similarly, a parenteral dosage form may contain smaller amounts of one or more of the active ingredients than an oral dosage form used to treat the same disease. See, e.g., Remington's Pharmaceutical Sciences, 18th ed., Mack Publishing, Easton Pa. (1990).

Whether a particular excipient is suitable for incorporation into a pharmaceutical composition or dosage form provided herein depends on a variety of factors, including, but not limited to, the route of administration. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, encompassed herein are pharmaceutical compositions and dosage forms that contain little, if any, lactose. As used herein, the term “lactose-free” means that the amount of lactose present, if any, is insufficient to substantially increase the degradation rate of an active ingredient.

Lactose-free compositions provided herein can comprise excipients that are listed, for example, in the U.S. Pharmacopeia (USP) 25 NF20 (2002). In certain embodiments, lactose-free compositions include active ingredients, a binder/filler, and a lubricant in pharmaceutically compatible and pharmaceutically acceptable amounts. In certain embodiments, lactose-free dosage forms include active ingredients, microcrystalline cellulose, pre-gelatinized starch, and magnesium stearate or stearic acid.

Further encompassed herein are anhydrous pharmaceutical compositions and dosage forms that include active ingredients, because water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance because moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that include lactose and at least one active ingredient that has a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, in certain embodiments, provided herein are anhydrous compositions packaged using materials to prevent exposure to water such that they can be included in suitable formulary kits. Examples of suitable packaging include, but are not limited to, hermetically sealed foils, plastics, unit dose containers (e.g., vials), blister packs, and strip packs.

Encompassed herein are pharmaceutical compositions and dosage forms that include one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients.

In certain embodiments, the dosage forms provided herein include Compound A, or an enantiomer or a mixture of enantiomers thereof, or a pharmaceutically acceptable salt, solvate, hydrate, co-crystal, clathrate, or polymorph thereof, in an amount ranging from about 0.10 to about 1000 mg, from about 0.10 to about 500 mg, from about 0.10 to about 200 mg, from about 0.10 to about 150 mg, from about 0.10 to about 100 mg, from about 0.10 to about 50 mg, from about 0.5 to about 10 mg, or from about 1 to about 5 mg. In certain embodiments, the dosage forms provided herein include Compound A, or an enantiomer or a mixture of enantiomers thereof, or a pharmaceutically acceptable salt, solvate, hydrate, co-crystal, clathrate, or polymorph thereof, in an amount of about 0.1, about 1, about 2, about 2.5, about 3, about 4, about 5, about 7.5, about 10, about 12.5, about 15, about 17.5, about 20, about 25, about 50, about 100, about 150, or about 200 mg. The dosage forms provided herein can include Compound A, or an enantiomer or a mixture of enantiomers thereof, or a pharmaceutically acceptable salt, solvate, hydrate, co-crystal, clathrate, or polymorph thereof in an amount described herein (e.g., 0.25, 0.5, 1, 2, 2.5, 3, 4, or 5 mg).

In certain embodiments, the dosage forms provided herein include Compound A, or an enantiomer or a mixture of enantiomers thereof, or a pharmaceutically acceptable salt, solvate, hydrate, co-crystal, clathrate, or polymorph thereof, and rituximab at a concentration of about 250 mg/m² to about 500 mg/m². The dosage forms provided herein can include Compound A, or an enantiomer or a mixture of enantiomers thereof, or a pharmaceutically acceptable salt, solvate, hydrate, co-crystal, clathrate, or polymorph thereof, and rituximab at a concentration of 375 mg/m² or 500 mg/m². The dosage forms provided herein can include Compound A, or an enantiomer or a mixture of enantiomers thereof, or a pharmaceutically acceptable salt, solvate, hydrate, co-crystal, clathrate, or polymorph thereof, and obinutuzumab at an amount of about 100 mg to about 1000 mg. The dosage forms provided herein can include Compound A, or an enantiomer or a mixture of enantiomers thereof, or a pharmaceutically acceptable salt, solvate, hydrate, co-crystal, clathrate, or polymorph thereof, and obinutuzumab at an amount of 100 mg. The dosage forms provided herein can include obinutuzumab at an amount of 900 mg. The dosage forms provided herein can include Compound A, or an enantiomer or a mixture of enantiomers thereof, or a pharmaceutically acceptable salt, solvate, hydrate, co-crystal, clathrate, or polymorph thereof, and obinutuzumab at an amount of 1000 mg.

In other embodiments, the dosage forms provided herein include Compound A, or an enantiomer or a mixture of enantiomers thereof, or a pharmaceutically acceptable salt, solvate, hydrate, co-crystal, clathrate, or polymorph thereof, and ibrutinib, or a pharmaceutically acceptable salt or solvate thereof, in an amount ranging from about 1 to about 1000 mg, from about 100 to about 800 mg, from about 100 to about 600 mg, from about 100 to about 500 mg, from about 140 to about 600 mg, from about 140 to about 500 mg, or from about 140 to about 420 mg. In certain embodiments, the dosage forms provided herein include Compound A, or an enantiomer or a mixture of enantiomers thereof, or a pharmaceutically acceptable salt, solvate, hydrate, co-crystal, clathrate, or polymorph thereof, in an amount described herein and ibrutinib, or a pharmaceutically acceptable salt or solvate thereof in an amount of about 100, about 140, about 280, about 420, or about 560 mg.

In one embodiment is a single dosage form that includes Compound A, or an enantiomer or a mixture of enantiomers thereof, or a pharmaceutically acceptable salt, solvate, hydrate, co-crystal, clathrate, or polymorph thereof at an amount of about 0.25, 0.5, 1, 2, 2.5, 3, 4, or 5 mg in combination with ibrutinib, or a pharmaceutically acceptable salt or solvate thereof at an amount of about 140, 280, 420, or 560 mg, thereby forming a single unit dosage form of Compound A, or an enantiomer or a mixture of enantiomers thereof, or a pharmaceutically acceptable salt, solvate, hydrate, co-crystal, clathrate, or polymorph thereof and ibrutinib, or a pharmaceutically acceptable salt or solvate thereof.

The single unit dosage form of Compound A, or a pharmaceutically acceptable salt, solvate, or stereoisomer thereof, and ibrutinib, or a pharmaceutically acceptable salt or solvate thereof may include Compound A, or a pharmaceutically acceptable salt, solvate, or stereoisomer thereof, at an amount of about 0.25, 0.5, 1, 2, 2.5, 3, 4, or 5 mg and ibrutinib, or a pharmaceutically acceptable salt or solvate thereof at an amount of 140 mg.

The single unit dosage form of Compound A, or a pharmaceutically acceptable salt, solvate, or stereoisomer thereof, and ibrutinib, or a pharmaceutically acceptable salt or solvate thereof may include Compound A, or a pharmaceutically acceptable salt, solvate, or stereoisomer thereof, at an amount of about 0.25, 0.5, 1, 2, 2.5, 3, 4, or 5 mg and ibrutinib, or a pharmaceutically acceptable salt or solvate thereof at an amount of 280 mg.

The single unit dosage form of Compound A, or a pharmaceutically acceptable salt, solvate, or stereoisomer thereof, and ibrutinib, or a pharmaceutically acceptable salt or solvate thereof may include Compound A, or a pharmaceutically acceptable salt, solvate, or stereoisomer thereof, at an amount of about 0.25, 0.5, 1, 2, 2.5, 3, 4, or 5 mg and ibrutinib, or a pharmaceutically acceptable salt or solvate thereof at an amount of 420 mg.

### Oral Dosage Forms

In certain embodiments, pharmaceutical compositions provided herein that are suitable for oral administration are formulated as discrete dosage forms, examples of which include, but are not limited to, tablets (e.g., chewable tablets), caplets, capsules, and liquids (e.g., flavored syrups). Such dosage forms contain predetermined amounts of active ingredients and may be prepared by some known methods of pharmacy. See generally, Remington's Pharmaceutical Sciences, 18th ed., Mack Publishing, Easton Pa. (1990).

In certain embodiments, the oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms may be prepared by some known methods of pharmacy. In certain embodiments, pharmaceutical compositions and dosage forms are prepared by uniformly and intimately admixing the active ingredients with liquid carriers, finely divided solid carriers, or both, and then shaping the product into the desired presentation if necessary.

In certain embodiments, a tablet is prepared by compression or molding. In certain embodiments, compressed tablets are prepared by compressing in a suitable machine the active ingredients in a free-flowing form, e.g., powder or granules, optionally mixed with an excipient. In certain embodiments, molded tablets are made by molding in a suitable machine a mixture of a powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms provided herein include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, AVICEL-PH-101, AVICEL-PH-103 AVICEL RC-581, AVICEL-PH-105 (FMC Corporation, American Viscose Division, Avicel Sales, Marcus Hook, Pa.), and mixtures thereof. An example of a specific binder is a mixture of microcrystalline cellulose and sodium carboxymethyl cellulose (e.g., AVICEL RC-581). Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. In certain embodiments, the binder or filler in pharmaceutical compositions provided herein is present in from about 50 to about 99 weight percent of the pharmaceutical composition or dosage form.

Disintegrants are used in the compositions provided herein to provide tablets the ability to disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation. In certain embodiments, the pharmaceutical compositions provided herein comprise from about 0.5 to about 15 weight percent or from about 1 to about 5 weight percent of disintegrant.

Disintegrants that are suitable for use in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that are suitable for use in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, but are not limited to, a syloid silica gel (AEROSIL200, W.R. Grace Co., Baltimore, Md.), a coagulated aerosol of synthetic silica (Degussa Co. of Plano, Tex.), CAB-O-SIL (a pyrogenic silicon dioxide, Cabot Co. of Boston, Mass.), and mixtures thereof. In certain embodiments, if used at all, lubricants are used in an amount of less than about 1 weight percent of the pharmaceutical compositions or dosage forms into which they are incorporated.

### Delayed Release Dosage Form

In certain embodiments, the active ingredients provided herein are administered by controlled release means or by delivery devices. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, each of which is incorporated herein by reference in its entirety. In certain embodiments, such dosage forms are used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Encompassed herein are single unit dosage forms suitable for oral administration, including, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Some suitable vehicles that can be used to provide parenteral dosage forms provided herein include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms provided herein. For example, cyclodextrin and its derivatives can be used to increase the solubility of a compound provided herein. See, e.g., U.S. Pat. No. 5,134,127, the disclosure of which is incorporated herein by reference in its entirety.

### Topical and Mucosal Dosage Forms

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed herein depend on the particular tissue to which a given pharmaceutical composition or dosage form will be applied. With that fact in mind, in certain embodiments, the excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Additional examples of such ingredients can be found, e.g., in Remington's Pharmaceutical Sciences, 16th and 18th eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Kits

In certain embodiments, compounds provided herein are not administered to a patient at the same time or by the same route of administration. Therefore, encompassed herein are kits which, when used by the medical practitioner, can simplify the administration of appropriate amounts of active ingredients to a patient.

In certain embodiments, a kit provided herein comprises a dosage form of a compound provided herein. In certain embodiments, the kit provided herein further comprises additional active ingredients. Examples of the additional active ingredients include, but are not limited to, those disclosed herein elsewhere.

In certain embodiments, the kit provided herein further comprises a device that is used to administer the active ingredients. Examples of such devices include, but are not limited to, syringes, drip bags, patches, and inhalers.

In certain embodiments, the kit provided herein further comprises cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Thus, in certain embodiments, the kit includes Compound A, or a pharmaceutically acceptable salt, solvate, or stereoisomer thereof at an amount described herein in an oral dosage form (e.g., tablet or capsule) and rituximab at a concentration described herein in a parental administration form (e.g., sterile intravenous solution).

The kits described herein can include Compound A, or a pharmaceutically acceptable salt, solvate, or stereoisomer thereof at an amount described herein in an oral dosage form (e.g., tablet or capsule) and obinutuzumab at an amount described herein in a parental administration form (e.g., sterile intravenous solution).

The kits described herein can include Compound A, or a pharmaceutically acceptable salt, solvate, or stereoisomer thereof at an amount described herein in an oral dosage form (e.g., tablet or capsule) and ibrutinib, or a pharmaceutically acceptable salt or solvate thereof, at an amount described herein in an oral dosage form (e.g., tablet or capsule).

### Clinical Trials Endpoints for Cancer Approval

“Overall survival” is defined as the time from randomization until death from any cause, and is measured in the intent-to-treat population. Overall survival should be evaluated in randomized controlled studies. Demonstration of a statistically significant improvement in overall survival can be considered to be clinically significant if the toxicity profile is acceptable, and has often supported new drug approval.

Several endpoints are based on tumor assessments. These endpoints include disease free survival (DFS), objective response rate (ORR), time to progression (TTP), progression-free survival (PFS), and time-to-treatment failure (TTF). The collection and analysis of data on these time-dependent endpoints are based on indirect assessments, calculations, and estimates (e.g., tumor measurements).

Generally, “disease free survival” (DFS) is defined as the time from randomization until recurrence of tumor or death from any cause. Although overall survival is a conventional endpoint for most adjuvant settings, DFS can be an important endpoint in situations where survival may be prolonged, making a survival endpoint impractical. DFS can be a surrogate for clinical benefit or it can provide direct evidence of clinical benefit. This determination is based on the magnitude of the effect, its risk-benefit relationship, and the disease setting. The definition of DFS can be complicated, particularly when deaths are noted without prior tumor progression documentation. These events can be scored either as disease recurrences or as censored events. Although all methods for statistical analysis of deaths have some limitations, considering all deaths (deaths from all causes) as recurrences can minimize bias. DFS can be overestimated using this definition, especially in patients who die after a long period without observation. Bias can be introduced if the frequency of long-term follow-up visits is dissimilar between the study arms or if dropouts are not random because of toxicity.

“Objective response rate” (ORR) is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study. If available, standardized criteria should be used to ascertain response. A variety of response criteria have been considered appropriate (e.g., RECIST criteria) (Therasse et al., (2000) J. Natl. Cancer Inst, 92: 205-16). The significance of ORR is assessed by its magnitude and duration, and the percentage of complete responses (no detectable evidence of tumor).

“Time to progression” (TTP) and “progression-free survival” (PFS) have served as primary endpoints for drug approval. TTP is defined as the time from randomization until objective tumor progression; TTP does not include deaths. PFS is defined as the time from randomization until objective tumor progression or death. Compared with TTP, PFS is the preferred regulatory endpoint. PFS includes deaths and thus can be a better correlate to overall survival. PFS assumes patient deaths are randomly related to tumor progression. However, in situations where the majority of deaths are unrelated to cancer, TTP can be an acceptable endpoint.

As an endpoint to support drug approval, PFS can reflect tumor growth and be assessed before the determination of a survival benefit. Its determination is not confounded by subsequent therapy. For a given sample size, the magnitude of effect on PFS can be larger than the effect on overall survival. However, the formal validation of PFS as a surrogate for survival for the many different malignancies that exist can be difficult. Data are sometimes insufficient to allow a robust evaluation of the correlation between effects on survival and PFS. Cancer trials are often small, and proven survival benefits of existing drugs are generally modest. The role of PFS as an endpoint to support licensing approval varies in different cancer settings. Whether an improvement in PFS represents a direct clinical benefit or a surrogate for clinical benefit depends on the magnitude of the effect and the risk-benefit of the new treatment compared to available therapies.

“Time-to-treatment failure” (TTF) is defined as a composite endpoint measuring time from randomization to discontinuation of treatment for any reason, including disease progression, treatment toxicity, and death. TTF is not recommended as a regulatory endpoint for drug approval. TTF does not adequately distinguish efficacy from these additional variables. A regulatory endpoint should clearly distinguish the efficacy of the drug from toxicity, patient or physician withdrawal, or patient intolerance.

In cancer research, the gold standard is to increase overall survival and PFS. In some embodiments, the benefits of the present invention are apparent when endpoints are measured as an increase in overall survival as compared to other arms. In some embodiments, the benefits of the present invention are apparent when endpoints are measured as an increase in PFS as compared to other arms.

In certain embodiments, the treatment of CLL may be assessed by the International Workshop Guidelines for CLL (see Hallek M, Cheson B D, Catovsky D, et al. Guidelines for the diagnosis and treatment of chronic lymphocytic leukemia: a report from the International Workshop on Chronic Lymphocytic Leukemia updating the National Cancer Institute-Working Group 1996 guidelines, Blood, 2008; (111) 12: 5446-5464) using the response and endpoint definitions shown therein and in particular:

| | Parameter | | |
|---|---|---|---|
| | CR | PR | PD |
| Group A | | | |
| Lymphadenopathy [symbol] | None >1.5 cm | Decrease ≧50% | Increase ≧50% |
| Hepatomegaly | None | Decrease ≧50% | Increase ≧50% |
| Splenomgaly | None | Decrease ≧50% | Increase ≧50% |
| Blood lymphocytes | <4000/μL | Decrease ≧50% from | Increase ≧50% over |
| | | baseline | baseline |
| Marrow [symbol] | Normocellular, <30% | 50% reduction in | |
| | lymphocytes, no B- | marrow infiltrate, or | |
| | lymphoid nodules. | B-lymphoid nodules | |
| | Hypocellular marrow | | |
| | defines CRi (5.1.6). | | |
| Group B | | | |
| Platelet count | >100,000/μL | >100,000/μL or | Decrease of ≧50% |
| | | increase ≧50% over | from baseline |
| | | baseline | secondary to CLL |
| Hemoglobin | >11.0 g/dL | >11 g/dL or increase | Decrease of >2 g/dL |
| | | ≧50% over baseline | from baseline |
| | | | secondary to CLL |
| Neutrophils [symbol] | >1500/μL | >1500/μL or >50% | |
| | | improvement over | |
| | | baseline | |

Group A criteria define the tumor load; Group B criteria define the function of the hematopoietic system (or marrow). CR (complete remission): all of the criteria have to be met, and patients have to lack disease related constitutional symptoms; PR (partial remission): at least two of the criteria of group A plus one of the criteria of group B have to be met; SD is absence of progressive disease (PD) and failure to achieve at least a PR; PD: at least one of the above criteria of group A or group B has to be met. Sum of the products of multiple lymph nodes (as evaluated by CT scans in clinical trials, or by physical examination in general practice). These parameters are irrelevant in some response categories.

Biologics and Biosimilar Anti-CD20 antibodies

In some embodiments, the anti-CD20 antibody is a molecule that is and has been approved as a biosimilar to one or more of the anti-CD20 antibodies specifically identified herein, including but not limited to rituximab, ibritumomab, tiuxetan, tositumomab, ofatumumab, ocaratuzumab, ocrelizumab and veltuzumab. The approval as a biosimilar may, for example, have been by one or more of the following jurisdictions: the United States of America, Canada, the European Union or any country within the European Union, and Japan.

### EXAMPLES

The examples below are carried out using standard techniques that are well known and routine to those of skill in the art, except where otherwise described in detail. The examples are intended to be merely illustrative.

### Example 1

In vitro, the immunomodulatory activity of Compound A on human NK cell function showed that Compound A dose dependently enhanced NK cell IFN-γ production in response to immobilized Ig-G and IL-2 stimulation (EC50=0.0015 μM). Without being bound by a particular theory, Compound A is believed to enhance the human NK cell killing function, antibody-dependent cellular cytotoxicity (ADCC), in rituximab coated lymphoma cell lines.

In vivo, Compound A administered with rituximab induced dose-dependent tumor growth inhibition in WSU-DLCL2 and DoHH2 lymphoma xenograft models. In the DoHH2 xenograft, there was modest dose-dependent tumor growth inhibition of Compound A when tested as a single agent at 3 and 30 mg/kg. Compound A in combination with rituximab in the DoHH2 xenograft significantly and dose-dependently delayed tumor growth. In the WSU-DLCL2 study, there were minimal effects with Compound A as a single agent. In combination with rituximab, Compound A at 3 and 30 mg/kg induced complete regressions in 60% and 90% of animals, respectively.

In pre-clinical models of DLBCL, lenalidomide and ibrutinib synergistically kill DLBCL cell lines. This synergy is explained by both drugs independently down-regulating interferon regulatory factor (IRF)4 (Yang, 2012). Compound A is also believed to down-regulate IRF4 in DLBCL cell lines. Ibrutinib also has immune modulatory effects on T-cells via inhibition of ITK, which ultimately promotes a T helper type 1 (Th1) T-cell phenotype in vitro and in vivo. These changes are complementary to the immune modulating effects of lenalidomide and possibly Compound A. It is possible that blocking BTK function in conjunction with Compound A may act synergistically in CLL.

In vitro combination efficacy studies were performed with Compound A and obinutuzumab (GA101). Two follicular (RL and DoHH2) and two diffuse large B cell lymphoma (DLBCL) (Oci-Ly10, WSU-DLCL2) cell lines were treated with either single agent Compound A (200 nM), single agent obinutuzumab (0 to 10,000 ng/mL) or in combination in 8-day cytotoxicity assays (cell titer glo). In both the single agent and combination studies, the cells were treated with Compound A for 8 days and with obinutuzumab treatment on the last 24 hours in the combination studies. Data indicate that Compound A and GA101 are synergistic in follicular lymphoma (FL) lines and additive in DLBCL cell lines with regards to cytotoxicity.

### Example 2

Study Design

Compound A administered orally to subjects with relapsed/refractory CLL/SLL. The following are evaluated:
Arm B: Compound A in combination with rituximab
Arm C: Compound A in combination with ibrutinib
Arm D: Compound A in combination with obinutuzumab

The study is conducted using a 3+3 dose escalation design to evaluate the safety of the combinations of a fixed dose of Compound A and rituximab, a fixed dose of Compound A and ibrutinib, and a fixed dose of Compound A and obinutuzumab to determine the NTD and MTD.

Intra-subject dose escalation schedules for the combinations of Compound A and rituximab, Compound A and ibrutinib, and Compound A and obinutuzumab are evaluated. These data are used to establish a RP2D for the combinations of Compound A and rituximab (Arm B), Compound A and ibrutinib (Arm C), and Compound A and obinutuzumab (Arm D).

Preliminary efficacy is evaluated and Compound A plasma PK is characterized for each subject enrolled to any of the Arms.

Once the RP2D is established for Arms B, C, and D, 2 expansion cohorts per arm are opened to further evaluate the safety and efficacy of Compound A in combination with rituximab, Compound A in combination with ibrutinib, and/or Compound A in combination with obinutuzumab:

Arm B (Compound A in combination with rituximab) (N=80)
Expansion Cohort 1 (N=40): Subjects who have not had prior treatment with ibrutinib
Expansion Cohort 2 (N=40): Subjects who have previously been treated with ibrutinib

Arm C (Compound A in combination with ibrutinib) (N=80):
Expansion Cohort 3 (N=40): Subjects with 17p deletion and/or p53 mutation
Expansion Cohort 4 (N=40): Subjects without 17p deletion and/or p53 mutation

Arm D (Compound A in combination with obinutuzumab) (N=80):
Expansion Cohort 5 (N=40): Subjects who have not had prior treatment with ibrutinib
Expansion Cohort 6 (N=40): Subjects who have previously been treated with ibrutinib

Subjects are treated until disease progression, unacceptable toxicity or discontinuation for any other reason.

### Example 3

Dose Escalation Phase: The Dose Escalation Phase evaluates ascending doses of Compound A.

Arm B (Compound A+rituximab):

Arm B1: Ascending starting doses of Compound A in combination with rituximab:

The starting dose of Compound A is 0.5 mg QD with subsequent cohorts evaluating dose escalations up to a maximum of 2.5 mg QD. Compound A is initiated on Cycle 1 Day 9. Rituximab, administered intravenously, is dosed at 375 mg/m² on Cycle 1 Days 1 and 8 and 500 mg/m² on Day 1 every other cycle thereafter up to Cycle 11 (Cycles 3, 5, 7, 9 and 11).

Arm B2, dose level 1: Starting dose of 0.5 mg QD Compound A with intra-subject dose escalation up to a maximum dose of 2.5 mg QD starting on Cycle 1 Day 9 combined with rituximab 375 mg/m² intravenously administered on Cycle 1 Days 1 and 8 and 500 mg/m² intravenously administered on Day 1 every other cycle thereafter up to Cycle 11 (Cycles 3, 5, 7, 9 and 11). Each subject is permitted to dose escalate based on individual subject tolerability.

Arm B2, dose level 2: Starting dose of 1.0 mg QD Compound A with intra-subject dose escalation up to a maximum dose of 2.5 mg QD starting on Cycle 1 Day 9 combined with rituximab 375 mg/m² intravenously administered on Cycle 1 Days 1 and 8 and 500 mg/m² intravenously administered on Day 1 every other cycle thereafter up to Cycle 11 (Cycles 3, 5, 7, 9 and 11). Each subject is permitted to dose escalate based on individual subject tolerability.

Compound A is administered daily starting at Cycle 1 Day 9 until disease progression, unacceptable toxicity, or discontinuation for any other reason.

Arm C (Compound A+ibrutinib):

Arm C1: Ascending starting doses of Compound A in combination with ibrutinib: The starting dose of Compound A is 0.5 mg QD (or as otherwise determined in Arm A) with subsequent cohorts evaluating dose escalations up to a maximum of 2.5 mg QD. Compound A is initiated on Cycle 2 Day 1. Ibrutinib 420 mg QD is administered starting on Cycle 1 Day 1.

Arm C2, dose level 1: Starting dose of 0.5 mg QD Compound A with intra-subject dose escalation up to a maximum dose of 2.5 mg QD starting on Cycle 2 Day 1 combined with ibrutinib 420 mg QD starting on Cycle 1 Day 1. Each subject is permitted to dose escalate based on individual subject tolerability.

Arm C2, dose level 2: Starting dose of 1.0 mg QD Compound A with intra-subject dose escalation up to a maximum dose of 2.5 mg QD starting on Cycle 2 Day 1 combined with ibrutinib (420 mg QD) starting on Cycle 1 Day 1. Each subject is permitted to dose escalate based on individual subject tolerability.

Compound A and ibrutinib are administered daily until disease progression, unacceptable toxicity, or discontinuation for any other reason.

Arm D (Compound A+obinutuzumab):

Arm D1: Ascending starting doses of Compound A in combination with obinutuzumab: The starting dose of Compound A is 0.5 mg QD with subsequent cohorts evaluating dose escalations up to a maximum of 2.5 mg QD. Compound A is initiated on Cycle 1 Day 9. Obinutuzumab, administered intravenously, dosed at 100 mg on Cycle 1 Day 1, 900 mg on Cycle 1 Day 2 and 1000 mg on Cycle 1 Days 8 and 15. Obinutuzumab is administered at a dose of 1000 mg on Day 1 every cycle thereafter up to Cycle 6.

Arm D2, dose level 1: Starting dose of 0.5 mg QD Compound A with intra-subject dose escalation up to a maximum dose of 2.5 mg QD starting on Cycle 1 Day 9 combined with obinutuzumab intravenously administered at a dose of 100 mg on Cycle 1 Day 1, 900 mg on Cycle 1 Day 2 and 1000 mg on Cycle 1 Days 8 and 15. Obinutuzumab is administered at a dose of 1000 mg on Day 1 of every cycle thereafter up to Cycle 6. Each subject is permitted to dose escalate based on individual subject tolerability.

Arm D2, dose level 2: Starting dose of 1.0 mg QD Compound A with intra-subject dose escalation up to a maximum dose of 2.5 mg QD starting on Cycle 1 Day 9 combined with obinutuzumab intravenously administered at a dose of 100 mg on Cycle 1 Day 1, 900 mg on Cycle 1 Day 2 and 1000 mg on Cycle 1 Days 8 and 15. Obinutuzumab is administered at a dose of 1000 mg on Day 1 of every cycle thereafter up to Cycle 6. Each subject is permitted to dose escalate based on individual subject tolerability.

Compound A is administered daily starting at Cycle 1 Day 9 until disease progression, unacceptable toxicity, or discontinuation for any other reason.

The MTD is defined as the highest dose level below the NTD with 0 or 1 of 6 DLT evaluable subjects experiencing DLT during the specified DLT evaluation period. A dose level is declared the MTD when at least 6 subjects have been enrolled and <2 subjects have experienced a DLT at that dose level.

An intermediate dose (i.e., one between the NTD and the last dose level before the NTD) or additional subjects within any dose cohort may be required to determine the MTD and RP2D more precisely.

If DLTs are believed to be an issue as a result of continuous dosing, intermediate dosing schedules starting at or below the MTD established on the continuous dosing schedule and potentially escalating as tolerated may be explored.

Evaluation of Intra-subject Dose Escalation: Each subject is permitted to dose escalate based on individual subject tolerability.

Determination of RP2D:

A preliminary RP2D for Arms B, C, and D based on an integrated assessment of the safety, available PK and pharmacodynamics data, and preliminary efficacy information are performed. The RP2D selected does not exceed the MTD from the dose escalation cohorts.

Expansion Phase:

Arm B (Compound A in combination with rituximab) (N=80):
Expansion Cohort 1 (N=40): Subjects who have not had prior treatment with ibrutinib
Expansion Cohort 2 (N=40): Subjects who have previously been treated with ibrutinib

Arm C (Compound A in combination with ibrutinib) (N=80):
Expansion Cohort 3 (N=40): Subjects with 17p deletion and/or p53 mutation
Expansion Cohort 4 (N=40): Subjects without 17p deletion and/or p53 mutation

Arm D (Compound A in combination with obinutuzumab) (N=80):
Expansion Cohort 5 (N=40): Subjects who have not had prior treatment with ibrutinib
Expansion Cohort 6 (N=40): Subjects who have previously been treated with ibrutinib

Compound A Dose

The first-in-human study of Compound A is being conducted in a broad tumor population including diverse solid tumors, lymphoma and MM. Part A of the study used a Compound A starting dose of 0.5 mg QD on a continuous dosing schedule. Using allometric scaling, this dose represented a dose ˜20-fold below the highest non-severely toxic dose (HNSTD) in primates based on preclinical toxicology studies. Part A dose escalation proceeded in 0.5 mg increments (0.5, 1.0, 1.5, 2.0, 2.5, 3.0, 3.5 mg) and established the nontolerated dose (NTD) at 3.5 mg QD and the MTD at 3.0 mg QD. Dose limiting toxicities (DLT) observed at 3.5 mg QD included Grade 3 fever and fatigue and Grade 3 muscle weakness. No DLTs were observed at doses of 3.0 mg QD or lower. In Part B, parallel expansion cohorts (14 to 20 subjects each) in 4 tumor indications (MM, NHL, HCC and gliomas) were subsequently enrolled at a dose of 3.0 mg QD. Given that subjects with CLL may be more sensitive to the side effects of immunomodulatory agents (IMiDs®) and other cereblon-binding agents, and the clinical safety, PK and pharmacodynamics data observed with Compound A to date, a lower Compound A starting dose of 0.25 mg QD was used in a CLL population.

A second consideration for the starting dose of Compound A is based on the relative in vitro potency between Compound A and lenalidomide and the relative human safety and tolerability of lenalidomide in CLL. Pre-clinical data indicate that Compound A is ˜10-fold more potent than lenalidomide in terms of immunomodulatory and anti-proliferative activity against a panel of lymphoma and MM cell lines. In primary cultures of human CLL cells, Compound A was ˜5-fold more potent than lenalidomide in 70% of samples tested. Clinical exploration of lenalidomide in CLL has established 5 mg to be a tolerable starting dose.

Dose Schedule

Initial dosing of Compound A is on a continuous daily dosing schedule (28 of 28 day cycles) based on the experience with lenalidomide in CLL. Intermittent dosing schedules of lenalidomide (21 of 28 day cycles) were explored in CLL in an attempt to mitigate the risk of neutropenia, but resulted in rebound lymphocytosis with an increase in circulating CLL cells during the 7 day drug holidays (Chen, 2011). As a result, most investigators have settled on continuous lenalidomide dosing schedules in this disease to allow for better disease control. The Compound A-CLL-001 protocol allows for the possibility of evaluating an intermittent dosing schedule of Compound A following review of preliminary safety, PK, pharmacodynamics and efficacy data, in particular to mitigate the expected risk of neutropenia.

Dose Escalation Strategy

Cohorts evaluating a fixed dose of Compound A starting at 0.5 mg in combination with rituximab, ibrutinib, or obinutuzumab are initiated. Subsequent cohorts evaluate ascending doses of Compound A in 0.5 mg increments up to a maximum of 2.5 mg in combination with rituximab (Arm B1), ibrutinib (Arm C1) and obinutuzumab (Arm D1).

It is anticipated that the combination of Compound A plus rituximab or obinutuzumab might also be associated with a lower risk of tumor flare allowing for higher doses of Compound A to be tolerated with this combination.

### Example 4

The screening period begins on the date the Informed Consent Document (ICD) is signed and lasts for up to 28 days. The ICD must be obtained prior to beginning any assessments solely for the purpose of this study. Standard of care assessments performed prior to signing the ICD (as described in the protocol) may be used for this study, assuming they meet the protocol requirements and following discussion with the sponsor's medical monitor. Recording of AEs/serious adverse events (SAEs) begins once the subject has signed the ICD. All prior anticancer treatments (e.g., chemotherapy, surgeries) and medical history, including approximate dates of treatment or diagnosis, must be recorded during screening.

Chronic lymphocytic leukemia-specific international prognostic classification reflecting the subject's status at the time of enrollment is documented according to the Binet or Rai Classification systems (Binet, 1981; Rai, 1987). Subjects with SLL are assessed according to the lymphoma guidelines used in the art.

Peripheral blood for fluorescent in situ hybridization (FISH), stimulated karyotype, Zeta-Chain-Associated Protein Kinase (ZAP 70), immunoglobulin heavy chain (IgVH) mutational status, p53 mutational status, and CD38 analyses are performed at screening. If a subject is rescreened, the FISH analyses (including 11q, 17p, 13q, trisomy 12, bc1-6, myc, 2p and 14q), stimulated karyotype and the biomarker analyses (IgVH mutational status, p53 mutational status, and ZAP 70) that were performed at the original screening visit do not need to be repeated if these FISH and biomarker analyses were done within 84 days of Day 1 of the study.

For patients in Arm C, in the absence of local laboratory results for 17p deletion and p53 mutation, central laboratory results are used to support enrollment decisions. Results for 17p deletion and p53 mutation are obtained within 84 days of Cycle 1 Day 1.

In addition, a comprehensive mutational panel for recurrent aberrations in CLL is performed.

Vital sign measurements are obtained at each visit and include systolic and diastolic blood pressure, heart rate, respiration rate, body temperature and body weight. Measurements of height are recorded only at the screening visit.

Complete physical examination, including evaluation of lymph nodes, spleen and liver are performed during screening, on Cycle 1 Day 1 and on Day 1 of each subsequent treatment cycle, at the EOT visit and at 28 days after the last IP discontinuation. Measurements of lymph nodes and documentation of any enlargement of the spleen and/or liver are recorded in the source document and eCRF.

Subjects undergo laboratory monitoring of plasma troponin-T and B-type natriuretic peptide (BNP) to evaluate for potential early evidence of cardiac toxicity. Testing is performed at screening, weekly during the first cycle of Compound A treatment and for the first cycle of each dose escalation (Days 1, 8, 15, and 22), Day 1 of each subsequent cycle, and at the EOT visit. A significant elevation of troponin-T is defined as a value greater than the upper limit of normal (ULN) for the assay with associated elevation of BNP or other significant cardiac symptoms or findings. A significant elevation of BNP is defined as a ≧20% increase over baseline with an absolute value >100 pg/mL. Subjects found at Screening to have baseline troponin-T>ULN or BNP>100 pg/mL must have baseline evaluation by a cardiologist during screening to optimize cardio-protective therapy.

Triplicate or single standard 12-lead ECGs are collected as follows:
Arm B: Cycle 1 Day 1: 0 and 1.5 hours (±15 minutes) after rituximab dosing Cycle 1 Day 9 and Cycle 1 Day 22: 0 and 1.5 hours (±15 minutes) after Compound A dosing
Arm C: Cycle 1 Day 1: 0 and 1.5 hours (±15 minutes) after ibrutinib dosing Cycle 2 Day 15: 0 and 1.5 hours (±15 minutes) after Compound A dosing
Arm D: Cycle 1 Day 1: 0 and 1.5 hours (±15 minutes) after obinutuzumab dosing Cycle 1 Day 9 and Cycle 1 Day 22: 0 and 1.5 hours (±15 minutes) after Compound A dosing

Left ventricular ejection fraction (LVEF) MUGA, or echocardiogram (ECHO) are conducted at screening, every 3 cycles (±7 days) (Cycle 4 Day 1, Cycle 7 Day 1, etc.) and at the EOT if not performed within the previous 8 weeks.

The following laboratory assessments are performed at the Screening visit:
Amylase, lipase, fasting lipid profile, creatine kinase, thyroid-stimulating hormone (TSH), free T4 (fT4), immunoglobulins (IgG, IgM and IgA only), T-cell subsets.
CBC: hemoglobin, hematocrit, red blood cell count with indices, white blood cell count with absolute differential and platelet count.
Serum chemistry: albumin, total protein, bicarbonate, calcium, phosphorus, serum creatinine, serum urea/blood urea nitrogen (BUN), glucose (fasting), potassium, sodium, magnesium, chloride, total bilirubin, alkaline phosphatase, aspartate aminotransferase (AST or serum glutamic oxaloacetic transaminase [SGOT]), alanine aminotransferase (ALT or serum glutamate pyruvic transaminase [SGPT]), and lactate dehydrogenase [LDH] and uric acid.
C-reactive protein.
Direct antiglobulin test (local laboratory only).
Coagulation tests: prothrombin time, international normalized ratio, and partial thromboplastin time.
Urinalysis: dipstick, with microscopy in event of positive (1+ or greater) blood or protein and 24-hour collection for creatinine clearance and protein quantification in the event of 2+ or greater protein.
Virus Serology: hepatitis B antigen (HBsAg), hepatitis B core antibody (anti-HBc), and hepatitis C virus (HCV) RNA.

All subjects are monitored for TLS. Subjects in this study are monitored for tumor flare reaction (TFR). TFR is defined as a sudden and tender increase in the size of the disease bearing sites, including the lymph nodes, spleen and/or the liver often accompanied by low-grade fever, diffuse rash and in some cases increase in the peripheral blood lymphocyte counts. TFR is assessed according to NCI CTCAE (v 3.0).

### Example 5

Efficacy Assessments. The following efficacy assessments are performed at scheduled intervals.

Tumor imaging assessment by computed tomography (CT) scan of the neck, chest, abdomen and pelvis. Disease progression based on physical and/or laboratory tests at any time is confirmed by CT scan. Scans performed in accordance with the standard of care, and prior to signing the ICD, are used for the screening assessment if within 56 days of Cycle 1 Day 1. All imaging assessments are evaluated by a reader at the local institution.

A bone marrow aspirate may be collected at Screening and a bone marrow aspirate and biopsy are collected at the time of the CR/CRi confirmation visit.

Peripheral blood for MRD assessment is collected during screening and at the time of CR/CRi confirmation visit.

Each subject is assessed according to Eastern Cooperative Oncology Group (ECOG) Performance Status (PS) criteria.

B-symptom Evaluation. Assessment of the presence or absence of B-symptoms is performed at screening and on Day 1 for all cycles, at EOT and 28 days post last dose of IP. B-symptoms are defined as any one or more of the following disease-related symptoms or signs:
a) Unintentional weight loss >10% within the previous 6 months.
b) Significant fatigue (i.e., ECOG PS 2 or worse; cannot work or unable to perform usual activities).
c) Fevers of greater than 100.5° F. or 38.0° C. for 2 or more weeks without other evidence of infection.

d) Night sweats for more than 1 month without evidence of infection.

Response assessment for CLL is completed according to the updated IWCLL guidelines for the diagnosis and treatment of CLL. Subjects with SLL are assessed according to the lymphoma guidelines. Lymph node responses are assessed separately and independently of increases in blood lymphocyte counts.

Blood and tumor specimens for exploratory biomarkers are collected throughout the study and are analyzed for markers, which are important in the mechanism of action of, or could predict response or toxicity to, the IPs.

The objectives of these correlative analyses are:
To evaluate pharmacodynamics markers that are indicative of pharmacologic activity of Compound A
To evaluate biomarkers that may be predictive of response or toxicity to Compound A, or Compound A in combination with either rituximab, ibrutinib or obinutuzumab

Samples collected include:
Saliva at baseline for germ line DNA
Optional bone marrow biopsy at screening
Bone marrow aspirate at screening
Whole blood at the time points

### Example 6

Dose Escalation Phase. Intensive PK is performed in a minimum of 3 subjects at the first dose level in Arm C. Sparse PK sampling is performed in the remaining subjects. During the Dose Expansion Phase, intensive PK is performed in a minimum of 3 subjects in Treatment Arm C on Cycle 1, Day 1 and Cycle 2, Day 15.

Arm B: Compound A in Combination with Rituximab

Subjects in Arm B are administered Compound A orally in an uninterrupted, once-daily schedule. Compound A is initiated on Cycle 1 Day 9 and on Day 1 of each cycle thereafter until disease progression, unacceptable toxicity or discontinuation for any other reason. Each dose is taken in the morning with about 8 ounces of water, with the subject having fasted overnight (minimum of 6 hours). Breakfast is delayed until at least one hour after dosing on non-PK sampling days. On PK sampling days, breakfast is delayed until 3-hours after the Compound A dose. Compound A is taken up to 12 hours late if dosing is delayed on a single day; otherwise that day's dose is omitted.

Dosing of rituximab is a single IV infusion with administration at a dose of 375 mg/m² on Cycle 1 Days 1 and 8. Rituximab is administered at a dose of 500 mg/m² on Day 1 every other cycle thereafter up to Cycle 11 (Cycles 3, 5, 7, 9 and 11). Following the Cycle 11 infusion, rituximab is discontinued as part of this study treatment, however subjects may continue on study treatment with Compound A until disease progression, unacceptable toxicity or discontinuation for another reason. There are no scheduled dose reductions for rituximab. Subjects experiencing hypersensitivity reaction to rituximab are treated immediately in accordance with the package insert or SmPC. Pretreatment for rituximab is in accordance with the package insert, SmPC, or institutional standards.

Arm B1: Ascending doses of Compound A in combination with rituximab are evaluated. The dose of Compound A is 0.25 mg QD or 0.5 mg QD (or as otherwise determined in Arm A) with subsequent cohorts evaluating dose escalations up to a maximum of 2.5 mg QD. The dose levels evaluated are outlined in Table 1. Dose levels are evaluated sequentially in the order listed below.

TABLE 1
Dose Escalation: Arm B1

| Dose | Compound | Compound | |
|---|---|---|---|
| level | A (mg/day) | A (mg/day) | Rituximab |
| 1 | 0.25 | 0.5 | 375 mg/m² |
| 2 | 0.5 | 1.0 | Cycle 1 Days 1 and 8 |
| 3 | 1.0 | 1.5 | 500 mg/m² |
| 4 | 1.5 | 2.0 | Cycles 3, 5, 7, 9, and 11: |
| 5 | 2.0 | 2.5 | Day 1 of each cycle |
| 6 | 2.5 | n/a | |

Arm B2: Compound A with intra-subject dose escalation in combination with rituximab is evaluated. Starting doses of 0.5 mg QD and 1.0 mg QD Compound A with intra-subject dose escalation up to a maximum dose of 2.5 mg QD are evaluated.

TABLE 2
Dose Escalation: Arm B2

| | Compound A (mg/daily) | | | | | | |
|---|---|---|---|---|---|---|---|
| Dose | Starting | | | | | | |
| Level | dose | Esc-1 | Esc-2 | Esc-3 | Esc-4 | Esc-5 | Rituximab |
| −1 | 0.25 | 0.5 | 1.0 | 1.5 | 2.0 | 2.5 | 375 mg/m² Cycle 1 |
| 1 | 0.5 | 1.0 | 1.5 | 2.0 | 2.5 | n/a | Days 1 and 8 |
| 2 | 1.0 | 1.5 | 2.0 | 2.5 | n/a | n/a | 500 mg/m² |
| | | | | | | | Cycles 3, 5, 7, 9, |
| | | | | | | | and 11: Day 1 of |
| | | | | | | | each cycle |

Compound A is administered until disease progression, unacceptable toxicity or discontinuation for any other reason.

Arm C: Compound A in Combination with Ibrutinib

Subjects in Arm C are administered Compound A orally in an uninterrupted, QD schedule. Compound A is initiated on Cycle 2 Day 1 and on Day 1 of each cycle thereafter until disease progression, unacceptable toxicity or discontinuation for any other reason. Each dose is taken in the morning with about 8 ounces of water, with the subject having fasted overnight (minimum of 6 hours). Breakfast is delayed until at least one hour after dosing on non-PK sampling days. On PK sampling days, breakfast is delayed until 3-hours after the Compound A dose. Compound A is taken up to 12 hours late if dosing is delayed on a single day; otherwise that day's dose is omitted.

Dosing of ibrutinib is as described in the IMBRUVICA package insert or SmPC. Subjects take orally three 140 mg capsules of ibrutinib daily for a total daily dose of 420 mg. Ibrutinib is taken once daily at approximately the same time each day. The capsules are swallowed whole with water and subjects should not open, break or chew capsules. Ibrutinib is administered daily starting Cycle 1 Day 1 until disease progression, unacceptable toxicity or discontinuation for any other reason.

Arm C1: Ascending doses of Compound A in combination with ibrutinib are evaluated. The starting dose of Compound A is 0.25 mg QD or 0.5 mg QD with subsequent cohorts evaluating dose escalations up to a maximum of 2.5 mg QD.

TABLE 3
Dose Escalation: Arm C1

| Dose level | Compound A (mg/day) | Compound A (mg/day) | Ibrutinib |
|---|---|---|---|
| 1 | 0.25 | 0.5 | 420 mg daily |
| 2 | 0.5 | 1.0 | |
| 3 | 1.0 | 1.5 | |
| 4 | 1.5 | 2.0 | |
| 5 | 2.0 | 2.5 | |
| 6 | 2.5 | n/a | |

Compound A and ibrutinib are administered until disease progression, unacceptable toxicity or discontinuation for any other reason.

Arm C2: Compound A with intra-subject dose escalation in combination with ibrutinib is evaluated. Starting doses of 0.5 mg QD and 1.0 mg QD Compound A with intra-subject dose escalation up to a maximum dose of 2.5 mg QD is evaluated.

TABLE 4
Dose Escalation: Arm C2

| | Compound A (mg/daily) | | | | | | |
|---|---|---|---|---|---|---|---|
| | Starting | | | | | | |
| Dose Level | dose | Esc-1 | Esc-2 | Esc-3 | Esc-4 | Esc-5 | Ibrutinib |
| −1 | 0.25 | 0.5 | 1.0 | 1.5 | 2.0 | 2.5 | 420 mg/daily |
| 1 | 0.5 | 1.0 | 1.5 | 2.0 | 2.5 | n/a | |
| 2 | 1.0 | 1.5 | 2.0 | 2.5 | n/a | n/a | |

Compound A and ibrutinib are administered until disease progression, unacceptable toxicity or discontinuation for any other reason.

Arm D: Compound A in Combination with Obinutuzumab

Subjects in Arm D are administered Compound A orally in an uninterrupted, QD schedule. Compound A is initiated on Cycle 1 Day 9 and on Day 1 of each cycle thereafter until disease progression, unacceptable toxicity or discontinuation for any other reason. Each dose is taken in the morning with about 8 ounces of water, with the subject having fasted overnight (minimum of 6 hours). Breakfast is delayed until at least one hour after dosing on non-PK sampling days. On PK sampling days, breakfast is delayed until 3-hours after the Compound A dose. Compound A is taken up to 12 hours late if dosing is delayed on a single day; otherwise that day's dose is omitted.

Dosing of obinutuzumab is as described in the GAZYVA® package insert or GAZYVARO SmPC®. Obinutuzumab is administered as an IV infusion at a dose of 100 mg on Cycle 1 Day 1 and 900 mg on Cycle 1 Day 2 and 1000 mg on Cycle 1 Days 8 and 15. Obinutuzumab is administered at a dose of 1000 mg on Day 1 of Cycles 2 through 6.

Obinutuzumab is administered in either an inpatient or outpatient clinical setting. Full emergency resuscitation facilities must be immediately available in the event of a severe infusion reaction and subjects must be under close supervision of the investigator or appropriately trained staff during the infusions and the post-infusion period. All transfer procedures during preparation require strict adherence to aseptic techniques. For microbiological stability, the diluted obinutuzumab infusion solution is used immediately. If not used immediately, the solution may be stored in a refrigerator at 2° C. to 8° C. (36° F. to 46° F.) for up to 24 hours prior to use.

Subjects are premedicated prior to each infusion. Obinutuzumab is only administered as a slow IV infusion through a dedicated line and never as an IV push or bolus. Infusion pumps are used to control the infusion rate.

Arm D1: Ascending doses of Compound A in combination with obinutuzumab are evaluated. The starting dose of Compound A is 0.25 mg QD or 0.5 mg QD with subsequent cohorts evaluating dose escalations up to a maximum of 2.5 mg QD.

TABLE 5
Dose Escalation: Arm D1

| | Compound | Compound | |
|---|---|---|---|
| Dose level | A (mg/day) | A (mg/day) | Obinutuzumab |
| 1 | 0.25 | 0.5 | Cycle 1 Day 1: 100 mg |
| 2 | 0.5 | 1.0 | Cycle 1 Day 2: 900 mg |
| 3 | 1.0 | 1.5 | Cycle 1 Day 8 and 15: 1000 mg |
| 4 | 1.5 | 2.0 | Cycles 2-6, Day 1: 1000 mg |
| 5 | 2.0 | 2.5 | |
| 6 | 2.5 | n/a | |

Compound A is administered until disease progression, unacceptable toxicity or discontinuation for any other reason.

Arm D2: Compound A with intra-subject dose escalation in combination with obinutuzumab is evaluated. Starting doses of 0.5 mg QD and 1.0 mg QD Compound A (or 0.25 mg QD if 0.5 mg QD is declared the NTD) with intra-subject dose escalation up to a maximum dose of 2.5 mg QD is evaluated.

TABLE 6
Dose Escalation: Arm D2

| | Compound A (mg/daily) | | | | | | |
|---|---|---|---|---|---|---|---|
| Dose | Starting | | | | | | |
| Level | dose | Esc-1 | Esc-2 | Esc-3 | Esc-4 | Esc-5 | Obinutuzumab |
| −1 | 0.25 | 0.5 | 1.0 | 1.5 | 2.0 | 2.5 | Cycle 1 Day 1: |
| 1 | 0.5 | 1.0 | 1.5 | 2.0 | 2.5 | n/a | 100 mg |
| 2 | 1.0 | 1.5 | 2.0 | 2.5 | n/a | n/a | Cycle 1 Day 2: |
| | | | | | | | 900 mg |
| | | | | | | | Cycle 1 Day 8 |
| | | | | | | | and 15: 1000 mg |
| | | | | | | | Cycles 2-6, |
| | | | | | | | Day 1: 1000 mg |

Compound A is administered until disease progression, unacceptable toxicity or discontinuation for any other reason.

1. A method of treating, preventing, or managing lymphoma or leukemia, said method comprising administering to a subject in need thereof Compound A: 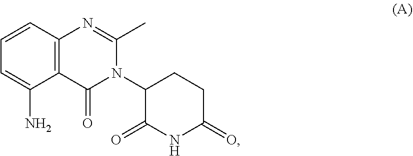 [SMILES: Cc1nc2cccc(N)c2c(=O)n1C1CCC(=O)NC1=O]
or a pharmaceutically acceptable salt or solvate thereof, in combination with an anti-cancer agent, wherein said anti-cancer agent is an anti-CD20 antibody, wherein said anti-CD20 antibody is rituximab or obinutuzumab.

2. (canceled)

3. The method of claim 1, wherein said anti-CD20 antibody is rituximab and said rituximab is administered to said subject on days 1 and 8 of a cycle at a concentration of 375 mg/m².

4. (canceled)

5. The method of claim 3, wherein said rituximab is further administered to said subject on day one of every other cycle in a 28-day cycle at a concentration of 500 mg/m².

6. (canceled)

7. (canceled)

8. The method of claim 5, wherein said subject was treated previously with rituximab.

9. (canceled)

10. The method of claim 5, wherein said subject was previously treated with ibrutinib, or a pharmaceutically acceptable salt or solvate thereof.

11. The method of claim 1, wherein said anti-CD20 antibody is obinutuzumab.

12. The method of claim 11, wherein said obinutuzumab is administered to said subject at an amount of 100 mg on day one of a cycle, 900 mg on day 2 of the cycle, and 1000 mg on days 8 and 15 of the cycle.

13. The method of claim 12, wherein said obinutuzumab is further administered to said subject on day one of every cycle in a 28-day cycle at an amount of 1000 mg.

14. (canceled)

15. (canceled)

16. The method of claim 1, wherein said subject was treated previously with obinutuzumab.

17. The method of claim 1, wherein said subject was not previously treated with ibrutinib, or a pharmaceutically acceptable salt or solvate thereof.

18. The method of claim 1, wherein said subject was previously treated with ibrutinib, or a pharmaceutically acceptable salt or solvate thereof.

19. (canceled)

20. (canceled)

21. (canceled)

22. (canceled)

23. (canceled)

24. (canceled)

25. The method of claim 1, wherein said lymphoma is selected from the group consisting of diffuse large B-cell lymphoma (DLBCL), primary CNS lymphoma (PCNSL), and non-Hodgkin's lymphoma (NHL).

26. The method of claim 25, wherein said lymphoma is non-Hodgkin's lymphoma (NHL) and the NHL is at least one of indolent, follicular lymphoma (FL), and aggressive.

27. (canceled)

28. (canceled)

29. (canceled)

30. (canceled)

31. The method of claim 1, wherein said leukemia is chronic lymphocytic lymphoma (CLL) or small lymphocytic lymphoma (SLL).

32. The method of claim 31, wherein said leukemia is CLL and comprises a 17p deletion or a p53 mutation.

33. (canceled)

34. (canceled)

35. (canceled)

36. The method of claim 31, wherein said leukemia is CLL, wherein said CLL is relapsed/refractory (r/r) CLL and said CLL is resistant to at least one anti-cancer agent or cancer therapy.

37. (canceled)

38. The method of claim 31, wherein said leukemia is SLL, wherein said SLL is relapsed/refractory (r/r) SLL and said SLL is resistant to at least one anti-cancer agent or cancer therapy.

39. (canceled)

40. (canceled)

41. (canceled)

42. The method of claim 1, wherein Compound A, or a pharmaceutically acceptable salt or solvate thereof is administered once daily for 5 days, followed by 2 days off in a 7 days cycle.

43. (canceled)

44. (canceled)

45. (canceled)

46. The method of claim 1, wherein Compound A, or a pharmaceutically acceptable salt or solvate thereof is administered in an amount of about 0.25 mg to about 5 mg per day.

47. (canceled)

48. (canceled)

49. (canceled)

50. (canceled)

51. (canceled)

52. (canceled)

53. (canceled)

54. The method of claim 1, wherein said subject is administered an ascending dose of Compound A, or a pharmaceutically acceptable salt or solvate thereof over the duration of treatment.

55. (canceled)

56. (canceled)

57. (canceled)
